(19) United States
(12) Patent Application Publication
(10) Pub. No.: US 2010/0041746 A1
(43) Pub. Date: Feb. 18, 2010

(54) NOVEL USE OF ORGANIC COMPOUNDS

(76) Inventors: Daniel D'Orazio, Halten (CH); Daniel Raederstorff, Flaxlanden (FR); Goede Schueler, Eimeldingen (DE); Ying Wang-Schmidt, Stallikon (CH); Karin Wertz, Rheinfelden (DE); Swen Wolfram, Waldshut-Tiengen (DE)

Correspondence Address: NIXON & VANDERHYE, PC, 901 NORTH GLEBE ROAD, 11TH FLOOR, ARLINGTON, VA, 22203, US

(21) Appl. No.: 12/301,505

(22) PCT Filed: May 24, 2007
(86) PCT No.: PCT/EP2007/004627

§ 371 Date: Oct. 8, 2009

(30) Foreign Application Priority Data
May 24, 2006 (EP) 06010723.2

(51) Int. Cl. A61K 31/352; A61K 31/12; A61K 31/36; C07C 49/255; C07D 317/46; C07D 311/02; A61P 21/00

(52) U.S. Cl. 514/456; 514/679; 514/466; 568/325; 549/438; 549/406

(57) ABSTRACT

The present invention refers to compounds of the general formulae Ia to Ie as defined above for use as/in a composition (especially foods and dietary supplements, cosmetic, as well as pharmaceutical compositions) for the prevention and improvement of muscular disorders and for the improvement of muscle function. Other fields of use are disorders connected to impaired lipid metabolism, impaired glucose metabolism and impaired insulin action, obesity, overweight conditions, eating disorders such as bulimia and anorexia nervosa, cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, dermatitis, and allergy, respiratory disorders such as asthma, chronic obstructive pulmonary disease (COPD), allergic diseases, and/or bone disorders such as osteoporosis and osteopenia. The compounds may also be used for bodyshaping, for improving the muscle:fat ratio or for accelerating skin wound healing. The present invention is also directed to dietary compositions such as (fortified) food, beverages, (fortified) feed, food additives, beverage additives, feed additives, clinical nutrition, dietary supplements, functional food, functional feed and nutraceuticals and to pharmaceutical compositions containing such compounds, to methods of treating the above mentioned disorders/diseases in mammals including humans and to the compounds of the formula I themselves. Another object of the present invention is the use of such compounds for the manufacture of a composition for the treatment of such disorders/diseases as mentioned above.

The present invention refers to compounds of the general formulae I or Ie, especially to compounds of the general formulae Ia to Ig as defined below for use as medicament, especially for the treatment of muscular disorders and for the improvement of muscle function.

Other fields of use are disorders connected to impaired lipid metabolism, impaired glucose metabolism and impaired insulin action, obesity, overweight conditions, eating disorders such as bulimia and anorexia nervosa, cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, and dermatitis, allergy, respiratory disorders such as asthma, chronic obstructive pulmonary disease (COPD), allergic diseases, and bone disorders such as osteoporosis and osteopenia. The compounds may also be used for accelerating skin wound healing.

The present invention is also directed to dietary compositions such as (fortified) food, beverages, (fortified) feed, food additives, beverage additives, feed additives, clinical nutrition, dietary supplements, functional food, functional feed and nutraceuticals and to pharmaceutical and body care compositions containing such compounds, to methods for treating muscular disorders, to methods for the improvement of muscle function, to methods of treating disorders connected to impaired lipid metabolism, impaired glucose metabolism, impaired insulin action and to methods for treating other conditions such as obesity, overweight conditions, eating disorders such as bulimia and anorexia nervosa, cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, and dermatitis, allergy, to methods for accelerating skin wound healing, to methods for treating respiratory disorders such as asthma, chronic obstructive pulmonary disease (COPD), allergic diseases, and to methods for treating bone disorders such as osteoporosis and osteopenia in mammals including humans.

Another object of the present invention is the use of such compounds for the treatment of muscular disorders and for the improvement of muscle function, for the treatment of disorders connected to impaired lipid metabolism, impaired glucose metabolism, impaired insulin action and for the treatment of obesity, overweight conditions, eating disorders such as bulimia and anorexia nervosa, for the treatment of cardiovascular diseases, atherosclerosis, vascular diseases, heart failure,-inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, for the acceleration of skin wound healing, for the treatment of respiratory disorders such as asthma, chronic obstructive pulmonary disease (COPD) and allergic diseases, as well as for the treatment of bone disorders such as osteoporosis and osteopenia, as well as the compounds as defined and described in more detail below (with the preferences given there) for these indications/application fields.

A further object of the present invention is the use of such compounds for the manufacture of a composition for the treatment of muscular disorders and for the improvement of muscle function, for the treatment of disorders connected to impaired lipid metabolism, impaired glucose metabolism, impaired insulin action and for the treatment of obesity, overweight conditions, eating disorders such as bulimia and anorexia nervosa, for the treatment of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, for the acceleration of skin wound healing, for the treatment of respiratory disorders such as asthma, chronic obstructive pulmonary disease (COPD) and allergic diseases, as well as for the treatment of bone disorders such as osteoporosis and osteopenia.

The expression “treatment” hereby also encompasses co-treatment, control, prevention and improvement, as well as maintenance of a healthy state.

The expression “disorder” encompasses also diseases, as well as the individual subjective opinion that the current state needs to be improved.

We now found that compounds of the general formula I or Ie

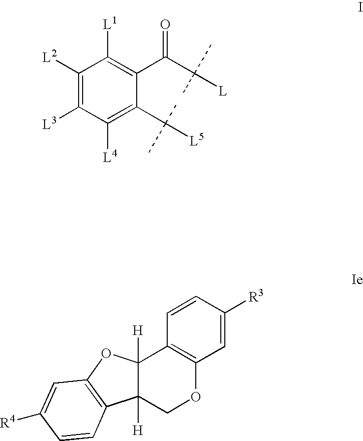

wherein L is either A or B when L⁵ is hydrogen or R⁵, or

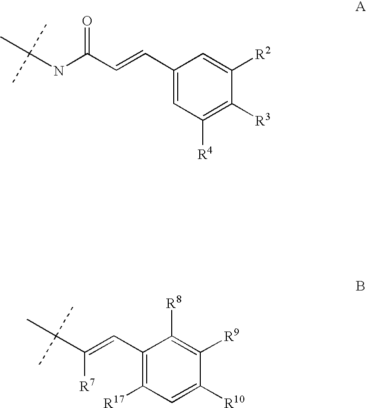

wherein L and L⁵ form the residue C or D,

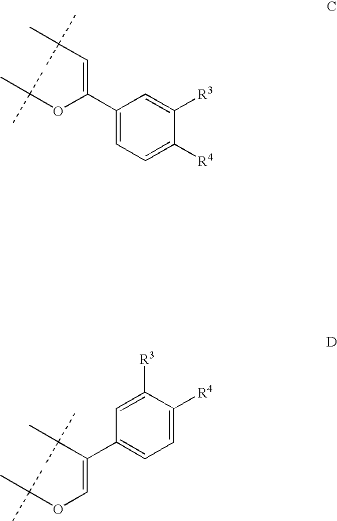

and wherein

L¹ is H, OH or R²; L² is H; L³ is H, OH or R⁶; L⁴ is H;

preferably L¹ is OH and L², L³ and L⁴ is H, when L is A;

preferably L¹, L² and L⁴ are H, L³ is R⁶ and L⁵ is R⁵ when L is B;

preferably L², L⁴ is H, L¹ is R² and L³ is R⁶ when L and L⁵ form together the residue C;

preferably L², L⁴ is H, L¹ is R² and L³ is R⁶ when L and L⁵ form together the residue D;

R² is H, OH or C₁₋₆-alkyloxy; R³, R⁴ and R⁶ are independently from each other OH or C₁₋₆-alkyloxy;

R⁵ is H or C₁₋₆-alkyloxy; R⁷ is H; or R⁵ and R⁷ are together —O—; R⁸, R¹⁰ is C₁₋₆-alkyloxy;

R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6; especially compounds of the formula Ia, Ib, Ic, Id and Ie

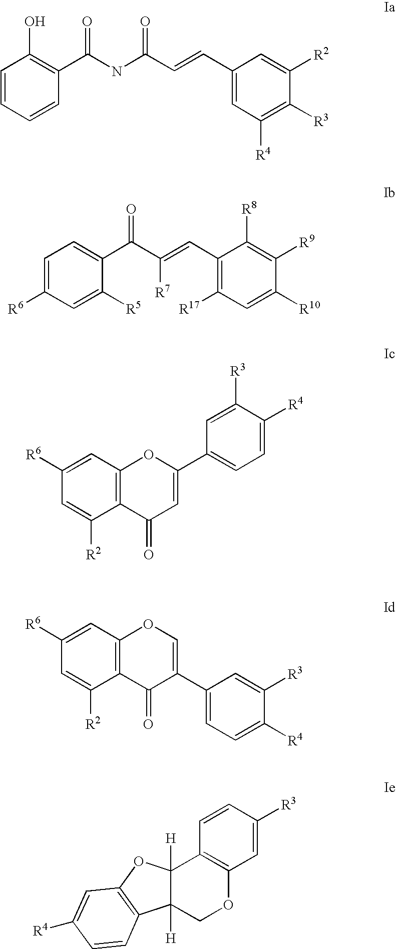

wherein

R² is H, OH or C₁₋₆-alkyloxy; R³, R⁴ and R⁶ are independently from each other OH or C₁₋₆-alkyloxy;

R⁵ is H or C₁₋₆-alkyloxy; R⁷ is H; or R⁵ and R⁷ are together —O—; R⁸ and R¹⁰ are independently from each other C₁₋₆-alkyloxy;

R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6; may be effective in the prevention, control, and/or treatment of muscular disorders and the improvement of muscle function and other fields of use such as the treatment/prevention of disorders connected to impaired lipid metabolism, of disorders connected to impaired glucose metabolism and/or impaired insulin action, the treatment/prevention of obesity, overweight conditions, eating disorders such as bulimia and anorexia nervosa, the treatment/prevention of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, and dermatitis, allergy, the acceleration of skin wound healing, the treatment/prevention of respiratory disorders such as asthma, chronic obstructive pulmonary disease (COPD), the treatment/prevention of allergic diseases, as well as the treatment/prevention of bone disorders such as osteoporosis and osteopenia.

The term “improvement of muscle function” encompasses the enhancement of the physical performance especially the enhancement of the physical endurance and the fatigue resistance.

Skeletal muscle fibers are generally classified as type I (oxidative/slow) or type II (glycolytic/fast) fibers. They display marked differences in respect to concentration, metabolism, and susceptibility to fatigue. Type I fibers are mitochondria-rich and mainly use oxidative metabolism for energy production, which provides a stable and long-lasting supply of ATP, and thus are fatigue-resistant. Type II fibers comprise three sub-types: IIa, IIx, and IIb. Type Ib fibers have the lowest levels of mitochondrial content and oxidative enzymes, rely on glycolytic metabolism as major energy source, and are susceptible to fatigue, while the oxidative and contraction functions of type Ia and IIx lie between type I and IIb. Adult skeletal muscle shows plasticity and can undergo conversion between different fiber types in response to exercise training or modulation of motoneuron activity (PLOS Biology 2004, 2(10), e294).

Determination of the muscle fiber composition in athletes revealed that elite endurance athletes have relatively more type I fibers than type II fibers in the trained musculature. Marathoners also tend to have more type I fibers. It was suggested that type I fiber might be a factor governing physical endurance capacity. In the contrary ageing and physical inactivity are conditions associated with a decrease in type I fibers, oxidative capacity and insulin sensitivity. It appears that the muscle oxidative capacity is a crucial factor for determining endurance and fatigue resistance. There seem to be an adaptive metabolic response of skeletal muscle to endurance exercise by controlling the number of oxidative muscle fibers (type I fibers).

The conversion of skeletal muscle fiber type IIb to type IIa and type I is regulated by different signaling pathways. For example the Ras/mitogen-activated protein kinase (MAPK), calcineurin, calcium/calmodulin-dependent protein kinase IV and the peroxisome proliferator γ coactivator 1 (PGC-1).

The compounds mentioned above may modulate these pathways and such may have an influence on the skeletal muscle fibers.

Such “diseases” connected to muscle disorders are muscle wasting and associated disorders such as sarcopenia, cachexia, muscular damage, muscular dystrophies and muscular fatigue. The term “treatment of muscle disorders” also encompasses the maintenance of muscle performance and/or strength and muscle function. Moreover, such compounds can improve endurance, as well as the muscle:fat ratio in individuals, who wish to do so, including also healthy individuals.

Muscle wasting is characterized by a progressive loss of muscle mass, weakening and degeneration of muscles especially the skeletal or voluntary muscles and the cardiac muscles. The processes by which atrophy and hypertrophy occur are conserved across mammalian species. Multiple studies have demonstrated that the same basic molecular, cellular, and physiological processes occur during atrophy in both rodents and humans.

Muscle wasting is due to a variety of causes and is associated with various pathologies, diseases and illnesses. These include but are not limited to muscular dystrophies caused by genetic disorders such as Duchenne's muscular dystrophy, progressive muscular dystrophy, Becker's type muscular dystrophy, Dejerine-Landouzy muscular dystrophy, Erb's muscular dystrophy, spinal muscular atrophy, and infantile neuroaxonal muscular dystrophy. Muscles wasting can also be caused by a variety of chronic diseases and the ageing process. As the body ages, an increasing proportion of skeletal muscle is replaced by fibrous tissue. Therefore, normal aging in humans is associated with progressive decrease in skeletal muscle mass and strength, a condition called sarcopenia, which contributes to frailty and falls.

Moreover, age related disorders such as hypertension, glucose intolerance and diabetes, obesity, dyslipidemia, atherosclerotic and cardiovascular disease are also associated with loss of muscle mass.

In addition other conditions such as cancer, autoimmune diseases, infections disease, HIV infection, AIDS, chronic inflammation, arthritis, malnutrition, renal diseases, chronic obstructive pulmonary disease (COPD), emphysema, osteomalacia, chronic lower back pain, peripheral nerve damage, spinal cord damage, chemical damage, central nervous system (CNS) damage are linked to or can cause muscle wasting. Finally, conditions resulting in muscle wasting may arise from disuse conditions such as long term immobilization due to illness or disability such as confinement in a wheelchair, prolonged bed rest, bone fracture or trauma. It is estimated that bed-rest after surgery causes loss of skeletal muscle mass of approximately 10% per week.

Untreated muscle wasting disorders can have serious health consequences. The changes that occur during muscle wasting can lead to a weakened physical state resulting in poor performance of the body and detrimental health effects.

Thus, muscle atrophy can seriously limit the rehabilitation of patients after immobilizations. Muscle wasting due to chronic diseases can lead to premature loss of mobility and increase the risk of disease-related morbidity. Muscle wasting due to disuse is an especially serious problem in elderly, who may already suffer from age-related deficits in muscle function and mass, leading to permanent disability and premature death as well as increased bone fracture rate. Despite the clinical importance of the condition few treatments exist to prevent or reverse the condition.

Muscle wasting is generally believed to result from disturbances in the energy or anabolic/catabolic pathways and is associated with chronic elevations in circulating inflammatory cytokines, in particular tumor necrosis factor alpha (TNF-alpha). Elevated levels of circulating inflammatory mediators, such as TNF alpha and interleukin 1 (IL-1), were believed to trigger the events leading to muscle wasting. Inflammatory mediators interfere with the function of satellite cells by decreasing or blocking their ability to fuse with or replace damaged myofibers, this action could ultimately result in the loss of skeletal muscle tissue.

The compounds described herein have anti-inflammatory activity partially mediated through a decrease in the production of inflammatory mediators such as TNF alpha and may be useful for the prevention and treatment of muscle wasting leading to muscle loss and atrophy and the associated muscle disorders in mammals, in particular humans.

Such diseases connected to impaired lipid metabolism are dyslipidemia and related lipid abnormalities such as hyperlipidemia, hypercholesteremia, hypertriglyceridemia and mixed dyslipidemia.

Dyslipidemia is characterized by abnormalities in circulating lipid levels due to alterations in lipid metabolism. These abnormalities can include any one or several of the different circulating lipid fractions (cholesterol, triglyceride, lipoprotein). Dyslipidemia includes hypercholesterolemia, which is an elevation of serum cholesterol above the normal limit (normal safe limit is approximately in the range of 125-200 mg/dl in human blood), hypertriglyceridemia which is an increase of serum triglycerides above the normal level (normal safe limit is approximately in the range of 30-140 mg/dl in human blood) and mixed lipid disorders. The blood cholesterol pool is generally dependent on dietary uptake of cholesterol from the intestine, and from the biosynthesis of cholesterol throughout the body, especially the liver. Triglycerides are synthesized in our body from the dietary fat especially when calorie intake exceeds the recommended levels.

In plasma, cholesterol and triglycerides are carried by protein-lipid particles called lipoproteins. Different classes of lipoproteins have been identified such as chylomicrons, chylomicron remnants, very low density lipoprotein (VLDL), intermediate-density lipids (IDLs), low density lipoprotein (LDL) and high density lipoprotein (HDL). These various types differ from one another in terms of size, density, and the amount of cholesterol, triglyceride, phospholipid, and apolipoprotein they contain. LDL, HDL, VLDL and chylomicron are the most often associated with dyslipidemia. Each lipoprotein performs a specific function in terms of the type of lipid it transports and the site to which they are transported. The majority of cholesterol in plasma is carried on apolipoprotein B-containing LDL and VLDL. Triglycerides are mainly carried by chylomicrons and VLDL. Based on their function LDL and VLDL are also called “bad cholesterol” while HDL are called “good cholesterol”. Therefore, when measuring cholesterol, it is important to measure its subfractions before making a diagnostics for lipid metabolic problems in particular LDL, HDL and VLDL.

Dyslipidemia includes hypertriglyceridemia and mixed dyslipidemia (hyperlipidemia). Hypertriglyceridemia involves a rise in the levels of VLDL, while mixed dyslipidemia (hyperlipidemia) involves a combination both hypertriglyceridemia and hypercholesterolemia and is also often associated with a drop in HDL levels. Thus, dyslipidemia is also a disorder of lipoprotein metabolism that results in an overproduction or a deficiency of lipoproteins. Dyslipidemia is typically characterized by any one or more of the following: elevated plasma triglycerides, elevated total plasma cholesterol, low High Density Lipoprotein cholesterol (HDL-c), elevated levels of Low Density Lipoprotein cholesterol (LDL-c). For example, dyslipidemia may be one or more of the following conditions: low HDL-c (<35 or 40 mg/dl), high triglycerides (>200 mg/dl), high LDL-c (>150 mg/dl), elevated cholesterol (>200 mg/dl). The manifestation of a dyslipidemia is often also defined according to national guidelines or experts recommendations. For example in the US the Third Report of the National Cholesterol Education Program (NCEP) Expert Panel on Detection, Evaluation, and Treatment of High Blood Cholesterol in Adults (Adult Treatment Panel III [ATP III]) defined the cholesterol levels. According to ATP III guidelines, a total serum cholesterol level of 200-239 mg/dL is considered “borderline high,” and a level greater than or equal to 240 mg/dL is considered “high.”

Dyslipidemia is widely considered as one of the main risk factor for cardiovascular vascular diseases (CVD) and atherogenesis. Cardiovascular disorders are among the leading causes of disability and death worldwide. High serum cholesterol, particularly cholesterol associated with LDL and VLDL, is one of the principal risk factors for atherogenesis. High triglycerides, increased small LDL, and decreased HDL levels all appear to be independently atherogenic. There is a strong inverse association between plasma HDL and the risk of CVD. A positive association exists between LDL cholesterol and risk of CVD. Thus, the risk of coronary artery disease increases when LDL and VLDL levels increase while high levels of cholesterol carried in HDL is protective against coronary artery disease. Triglycerides also seem to play an important role in CVD. High level of fasting triglycerides is a strong risk factor for ischaemic heart disease in elderly men independently of other major risk factors including HDL-cholesterol.

People with combined hyperlipidemia, which is characterized by elevated serum levels of both cholesterol and triglycerides, run a higher risk of heart disease than those with only a high LDL cholesterol level. Therefore, lowering both levels is a desired goal.

Hypercholesterolemia is usually treated with statins, which by inhibition of HMG-CoA reductase lowers the plasma concentration of LDL-c but have little effects on HDL-c. Fibrates are used to treat hypertriglyceridemia. However, relatively high doses of fibrates are needed, leading to drug side effects. Moreover, they induce only a modest HDL-c elevation.

It has been shown that lowering LDL-c is not sufficient for reducing the risk of cardiovascular disease in some patients, particularly those with normal LDL-c levels. However, there is no good drug treatment efficacious both in decreasing LDL-c and increasing HDL-c. Thus there is a need for new treatments which are able to decrease LDL-c and increase HDL-c. This need is fulfilled by the compounds of the formulae I and If, especially by the compounds of the formulae Ia to Ie of the present invention.

Such a disease connected to impaired glucose metabolism and impaired insulin action is diabetes mellitus, especially diabetes mellitus type 1 and 2, more especially (non-autoimmune) non-insulin dependent diabetes mellitus (NIDDM; so called Type 2 Diabetes). Another such disease is syndrome X.

Diabetes mellitus defines a complex of metabolic diseases derived from multiple causative factors and is characterized by impaired glucose metabolism, usually associated with impaired protein and fat metabolism. This results in elevated fasting and postprandial serum glucose that leads to complications if left untreated. Four different forms of diabetes mellitus are known, (1) type 1 diabetes mellitus, (2) type 2 diabetes mellitus, (3) the so-called gestational diabetes mellitus, which begins or is recognized for the first time during pregnancy, and (4) some other forms which are mainly based on genetic defects.

The term “diabetes mellitus” includes, but is not limited to, metabolic abnormalities such as increased blood glucose level, obesity associated pathologies, impaired glucose tolerance, increased insulin resistance, hyperlipidemia, dyslipidemia, increase in cholesterol (hypercholesterinemia, hypertriglycerinemia), hyperinsulinemia, hypertension, and microalbuminuria. Impaired glucose tolerance and impaired fasting glucose are the two symptoms referred to as pre-diabetes mellitus. This stage is associated with the so-called insulin resistance, one of a group of metabolic diseases called “syndrome X” or “metabolic syndrome”. Since type 2 diabetes mellitus is often associated with other symptoms from syndrome X, such as hypertriglyceridemia or dyslipidemia, the compounds according to the present invention are also useful for the treatment or prevention of syndrome X.

The two major forms of diabetes mellitus are the type 1 and type 2 diabetes mellitus, of which type 2 diabetes mellitus is the most prevailing form. Type 1 and type 2 diabetes mellitus are associated with hyperglycemia, hypercholesterolemia and hyperlipidemia. The insensitivity to insulin and absolute insulin deficiency in type 1 and 2 diabetes mellitus leads to a decrease in glucose utilization by the liver, muscle and the adipose tissue and to increased blood glucose levels. Uncontrolled hyperglycemia is associated with the dysfunction and failure of various organs such as the eyes, heart, blood vessels, kidney and nerves thus leading to increased and premature mortality due to an increased risk for microvascular and macrovascular diseases, including nephropathy, neuropathy, retinopathy, ulceration of the legs and feet, fatty liver disease, hypertension, cardiovascular diseases, and cerebrovascular diseases (stroke), the so-called diabetic complications.

Recent evidence showed that tight glycemic control is a major factor in the prevention of these complications in both type 1 and type 2 diabetes mellitus. Therefore, optimal glycemic control by drugs or therapeutic regimens is an important approach for the treatment of diabetes mellitus.

Type 1 diabetes mellitus is the form of diabetes mellitus which usually begins with childhood or puberty and is characterized by an auto-immune destruction of the insulin-producing β-cells leading to a complete deficiency of insulin secretion.

Type 2 diabetes mellitus is the form of diabetes mellitus which occurs predominantly in adults in whom adequate production of insulin is available in the early stage of the diseases, yet a defect exists in insulin sensitivity, especially in insulin-mediated utilization and metabolism of glucose in peripheral tissues. The changes in various tissues associated with type 2 diabetes mellitus exist even before clinical symptoms are detected.

Therapy of hyperlipidemia involves dietary and lifestyle changes, followed by pharmacological treatment. The major blood lipid lowering drugs include the statin family, niacin (in combination with statin), the fibrate family. The statin family of drugs can reduce LDL cholesterol by as much as 60 percent, depending upon the specific drug and the dose. Statins also reduce triglycerides and modestly increase HDL. Particularly at the initiation of therapy, the physician needs to monitor liver function. A rare complication of statin therapy is muscle damage. The development of muscle aches during treatment, therefore, is an indication to notify your physician. Examples of currently used statins in the United States are Lipitor, Zocor, Pravachol, Lescol and Creston. Niacin may be added to statin therapy. High dosages of niacin are particularly potent in raising HDL, and lowering triglycerides. On the other hand, its side effects can be annoying. In particular, niacin causes flushing. Niacin also causes an increase in blood sugar, and can be toxic to the liver. The fibrate family is particularly effective in lowering triglyceride. Some physicians consider fibrates to be the drug of choice for triglyceride levels in excess of 400 mg/dl. As the triglyceride levels fall, HDL frequently increases. Toxicity of the fibrates includes liver damage and muscle damage (a higher probability, when combined with statins). The most widely used fibrate in the United States currently is Lopid. Several agents reduce fat absorption from the gut, lowering blood cholesterol. The most commonly used agents are Zetia, Benecol, Welcol, Cholestyramine and Colestipol.

Therefore, there is a need for compounds with minimal side effects for the prevention, control and/or treatment of disorders connected to impaired lipid metabolism and hyperlipidemia and for the prevention of the physical complications associated with it/them as mentioned above. Many patients are interested in alternative therapies which could minimize the side effects and drug resistance associated with high-dose of drugs and yield additive clinical benefits. In addition, people at high risk to develop some symptoms of metabolic syndrome such as obese people, people with family history of Type 2 diabetes, and women with history of pregnancy diabetes, require early prevention measures. Therefore, there is also an increasing interest in the development of a dietary supplement that may be used to prevent the development of dyslipidemia in people at risk especially in elderly persons, but also in obese children.

We now found that the compounds of the general formulae I and Ie, especially compounds of the formulae Ia, Ib, Ic, Id and Ie as defined above, as well as compounds of the formula If and Ig as defined below may be effective agents in the prevention, control and/or treatment of muscular disorders, the improvement of muscle function and muscle performance, disorders connected to impaired lipid metabolism, impaired glucose metabolism and impaired insulin action.

Therapeutic effects of these compounds may include, but are not limited to, the following ones. Therefore, the present invention is directed to the use of the compounds of the general formulae I and Ie, especially compounds of the formulae Ia, Ib, Ic, Id and Ie, as defined above and compounds of the formula If and Ig as defined below (preferences: compounds 4 and 11>compounds 3, 4, 6 and 11>compounds of formula If and Ig and compound 11>compounds 1 to 6 and 11>compounds 1 to 8 and 11) for
increasing fitness, physical endurance and physical performance; i.e. humans or animals to which the compounds are administered may be able to perform physical activities for a longer time than humans or animals to which the compounds were not administered;
improving skeletal muscle endurance and resistance to fatigue, the compounds are muscle remodelling agents, increasing the proportion of type I oxidative muscle fibres and stimulating mitochondrial biogenesis thus increasing muscle oxidative capacity which is a key factor for muscle endurance and muscle fatigue resistance;
preventing muscle mass loss; inhibiting muscle catabolism and increasing muscle anabolism, enhancing muscle recovery, reducing protein loss due to chronic illness, reducing cachexia;
preventing sarcopenia; preventing frailty or age-related function decline due to aging, maintaining muscle strength and function in elderly mammals;
improving muscle dystrophy, improve muscle disorders caused by a defect in one or more genes that control muscle function, decrease wasting of skeletal muscle;
increasing muscle metabolism to boost energy mobilization;
improving skeletal muscle mass by stimulating anabolic pathways, inhibiting catabolic pathways and accelerating muscle regeneration when damaged;
lowering triglyceride levels in the blood; maintaining a healthy/normal blood lipid balance and a healthy/normal blood lipid profile by regulating/adjusting the blood lipid levels thus optimizing the blood lipid profile; treating elevated blood lipid levels and high blood cholesterol levels by metabolizing cholesterol and blood lipids; helping to reduce the cholesterol levels in a hyperlipidemic patient; improving dyslipidemia; i.e. the compounds of the formula I may be blood lipids lowering agents;
improving blood cholesterol profile in blood, by decreasing LDL and VLD cholesterol (“bad cholesterol) and increasing HDL cholesterol (“good cholesterol”);
being hypolipemic, i.e. preventing dyslipidemia by modulating blood lipid levels;
helping to prevent obesity; maintain optimal body weight, resist weight gain by creating a state of obesity resistance, resisting dietary (fat) induced obesity, being obesity resistant;
promoting fat burning; act as a regulator of fat burning, increasing energy expenditure by fatty acid oxidation, increasing fat metabolism, promoting fat oxidation and preventing fat induced obesity, decreasing body fat and increasing muscle mass;
supporting the fat loss achieved with diet and exercise by increasing the oxidative capacity of the body;
decreasing the storage of fat in the body of mammals, especially humans;
helping to achieve a good silhouette (body shaping), decreasing body fat and increasing lean muscle mass, preventing or decreasing overweight;
increasing thermogenesis; increasing the metabolism of a human or animal to burn more energy, protecting against obesity;
alleviating skin disorders, especially improving skin wound healing in conditions, where this is desirable;
reducing inflammation, and the associated symptoms,
maintaining energy homeostasis and lipid homeostasis to treat or inhibit the progression of for example dyslipidemia;
helping to manage blood sugar levels, i.e. helping the body by balancing the blood sugar levels; helping to keep balanced blood glucose levels, particularly in humans with diabetes; aiding by enhancing the glucose uptake by the cells and by reducing blood (circulating) sugar levels, thus improving or restoring the glucose tolerance; lowering the blood glucose level; optimizing the glycemic response; normalizing the glucose tolerance; i.e. the compounds of the formula I may be α-glucosidase inhibitors, hyperglycemia treating and/or controlling agents and blood glucose controlling agents;
reducing sweetness cravings;
preserving or improving the pancreatic β-cell function, thus promoting a healthy pancreatic function; i.e. the compounds of the formula I may be pancreatic β-cell function improvers;
treating or controlling the insulin resistance/sensitivity by e.g. helping to restore/enhance the insulin sensitivity in peripheral tissues, such as adipose, liver and skeletal muscle; i.e. the compounds of the formula I may be insulin sensitizers;
lowering insulin resistance;
delaying, preventing or controlling diabetes mellitus type 2, especially NIDDM, and dyslipidemia and thus preventing also the diabetes accompanying disorders/complications such as the ones mentioned above; i.e. the compounds of the formula I are diabetes type 2 preventing agents;
activating adipocytes, thus increasing insulin sensitivity;
repartioning of fat from lipolytic visceral fat depots into subcutaneous fat depots, thus decreasing the risk of obesity associated pathologies such as cardiovascular diseases;
reducing the circulation of free fatty acids (FFA), thus improving the insulin sensitivity in obese people;
maintaining endothelial function;
improving bone health and preventing bone-related disorders, such as osteoporosis and osteopenia.

Therefore, the present invention is directed to compounds of the general formula I and Ie

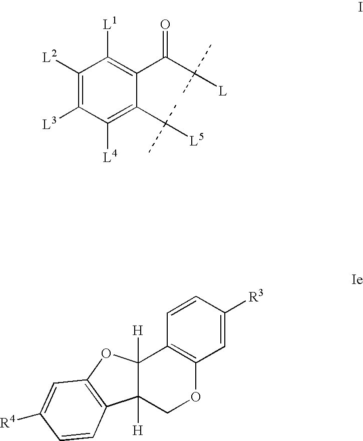

wherein L is either A or B when L⁵ is hydrogen or R⁵, or

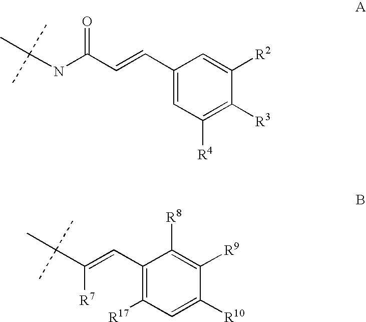

wherein L and L⁵ form the residue C or D,

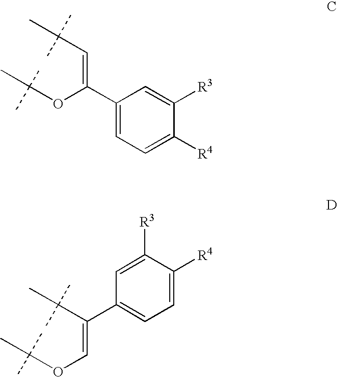

and wherein

L¹ is H, OH or R²; L² is H; L³ is H, OH or R⁶; L⁴ is H;

preferably L¹ is OH and L², L³ and L⁴ is H, when L is A;

preferably L¹, L² and L⁴ are H, L³is R⁶ and L⁵ is R⁵ when L is B;

preferably L², L⁴ is H, L¹ is R² and L³ is R⁶ when L and L⁵ form together the residue C;

preferably L², L⁴ is H, L¹ is R²and L³ is R⁶ when L and L⁵ form together the residue D;

R² is H, OH or C₁₋₆-alkyloxy; R³, R⁴ and R⁶ are independently from each other OH or C₁₋₆-alkyloxy;

R⁵ is H or C₁₋₆-alkyloxy; R⁷ is H; or R⁵ and R⁷ are together —O—; R⁸, R¹⁰ is C₁₋₆-alkyloxy;

R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6; for use as medicament, especially for the treatment of muscular disorders, the improvement of muscle function and muscle performance, as well as for the treatment of disorders connected to impaired lipid metabolism, impaired glucose metabolism and/or impaired insulin action.

Preferably the present invention is directed to compounds of the general formulae Ia to Ie

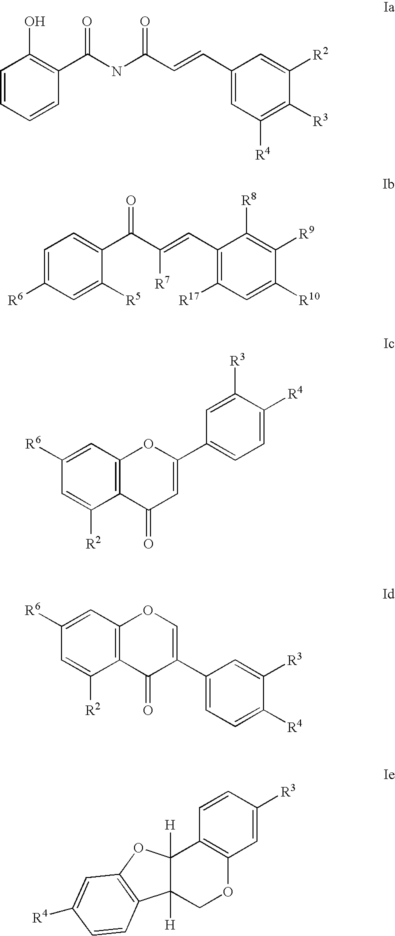

wherein

R² is H, OH or C₁₋₆-alkyloxy; R³, R⁴ and R⁶ are independently from each other OH or C₁₋₆-alkyloxy;

R⁵ is H or C₁₋₆-alkyloxy; R⁷ is H; or R⁵ and R⁷ are together —O—; R⁸ and R¹⁰ are independently from each other C₁₋₆-alkyloxy;

R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6; for use as medicament, especially for the treatment of muscular disorders, the improvement of muscle function and muscle performance, as well as for the treatment of disorders connected to impaired lipid metabolism, impaired glucose metabolism and impaired insulin action.

Furthermore, the compounds of the formulae I and Ie, especially the compounds of the formulae Ia to If, as defined above may also:
be anti-atherosclerotic, i.e. help preventing atherosclerosis; having antiatherogenic effects by modulating various process involved in atherosclerosis development for example increasing plasma HDL, decreasing lipid uptake in cell notably macrophages and decreasing pro-inflammatory markers;
be anti-inflammatory; i.e help to prevent inflammation;
promote skin health, especially prevent and/or attenuate psoriasis, acne, warts, hyperpigmentation, keratosis, ichthyosis, skin lesions, leukoplakia, rosacea, accelerate/improve skin wound healing.

Especially preferred for such uses are compounds of the formulae Ia to Ie, wherein
R² is hydrogen, hydroxyl or methoxy; and/or
R³ and R⁴ are independently from each other hydroxyl or methoxy; and/or
R⁶ is hydroxyl or methoxy; and/or
R⁵ is hydrogen or methoxy; and/or
R⁷ is hydrogen; or
R⁵ and R⁷ are together —O—; and/or
R⁸ and R¹⁰ are methoxy; and/or
R⁹ is hydrogen, methoxy or cinnamoyloxy; or
R⁹ and R¹⁰ form together a group O—(CH₂)ₓ—O with x=1 or 2; and/or
R¹⁷ is N-acetyl, N-methyl-2-aminoethyl.

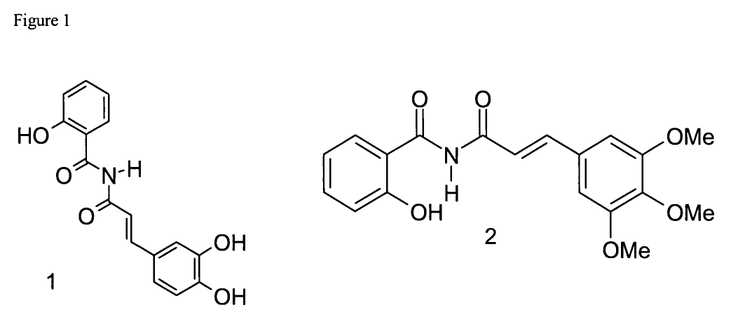

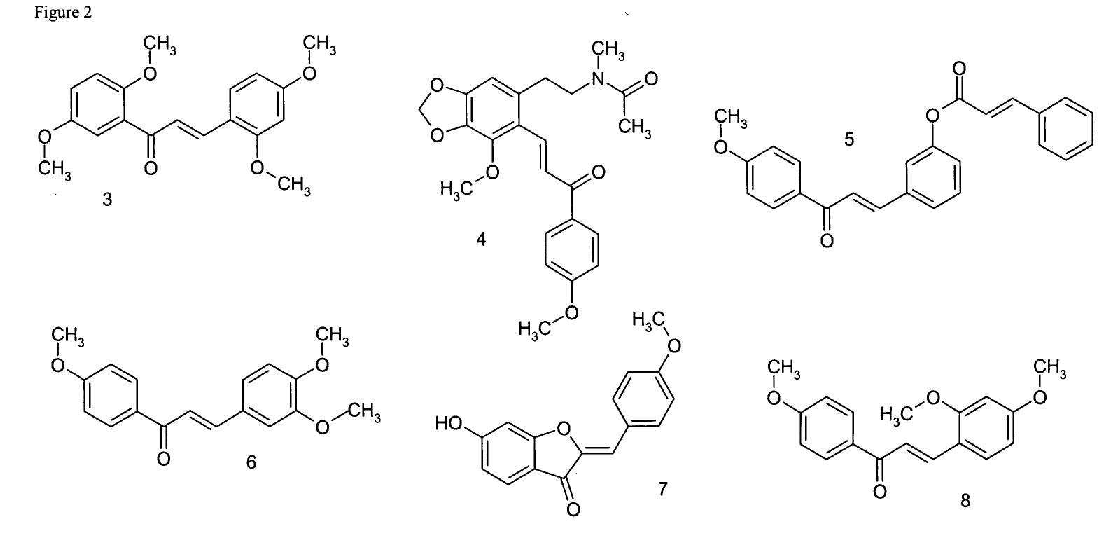

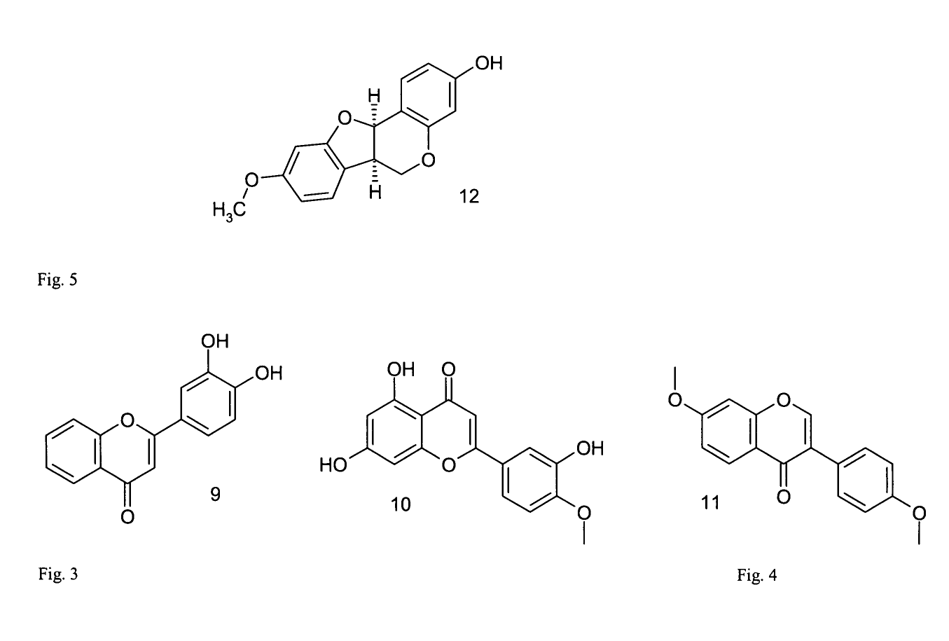

In an especially preferred embodiment of the present invention a compound selected from the group consisting of
the compound of the general formula Ia, wherein R1=R3=R4=OH and R2=H(=compound 1; see FIG. 1);
the compound of the general formula Ia, wherein R1=OH, R2=R3=R4=methoxy (=compound 2; see FIG. 1);
the compound of the general formula Ib, wherein R5=R6=R8=R10=methoxy and R7=R9=R17=H (=compound 3; see FIG. 2);
the compound of the general formula Ib, wherein R5=R7=H, R6=R8=methoxy, R9 and R10 form together —O—CH₂—O— and R17=N-acyl,N-methyl)-2-aminoethyl (=compound 4; see FIG. 2);
the compound of the general formula Ib, wherein R5=R7=R8=R10=R17=H, R6=methoxy and R9=cinnamyloxy (=compound 5; see FIG. 2); (CAS-No. 619313-14-3)
the compound of the general formula Ib, wherein R5=R7=R8=R17=H, R6=R9=R10=methoxy (=compound 6; see FIG. 2);
the compound of the general formula Ib, wherein R8=R9=R17=H, R6=OH, R5 and i R7 are together O, R10=methoxy (=compound 7; see FIG. 2); (CAS-No. 20727-61-1)
the compound of the general formula Ib, wherein R5=R7=R9=R17=H, R6=R8=R10=methoxy (=compound 8; see FIG. 2);
the compound of the general formula Ic, wherein R3=R4=OH, and R2=R6=H(=compound 9; see FIG. 3);
the compound of the general formula Ic, wherein R2=R3=R6=OH, and R4=methoxy (=compound 10; see FIG. 3);
the compound of the general formula Id, wherein R2=R3=H and R4=R6=methoxy (=compound 11; see FIG. 4);
the compound of the general formula Ie, wherein R3=R4=methoxy and the two hydrogens are cis to each other (=compound 12; see FIG. 5);

or mixtures thereof, are used.

Even more preferred are the compounds 1 to 8 and 11, then the compounds 1 to 6 and 11, then compound 11, the compounds of the general formula Ig as defined below and the compounds of the general formula If with X¹═H or CH₃ and X²═X³, X⁴ or X⁵, then the compounds 3, 4, 6 and 11 (compounds falling under general formula If with X¹═H or CH₃ and X²═X³, X⁴ or X⁵ and compounds falling under the general formula Ig). Most preferred are compounds 4 and 11, especially compound 11.

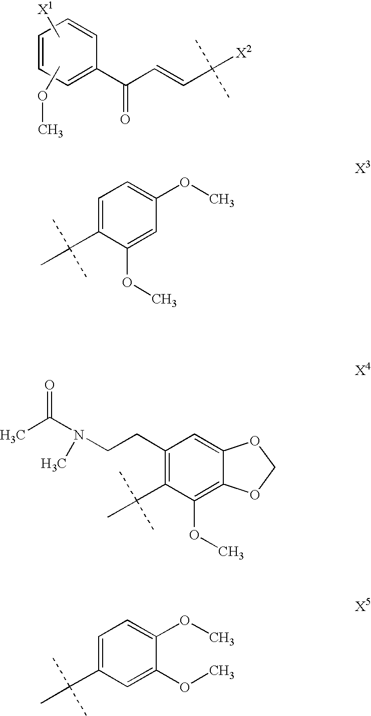

The term “compound of the formula Ia/Ib/Ic/Id/Ie/If/Ig” also encompasses any material or extract of a plant containing such a compound of the formula Ia/Ib/Ic/Id/Ie/If/Ig, preferably in an amount of at least 1 weight-% (except for the case of compound 1), more preferably in an amount of at least 50 weight-%, even more preferably in an amount of at least 90 lo weight-%, based on the total weight of the plant material or extract. The terms “material of a plant” and “plant material” used in the context of the present invention mean any part of a plant.

The compound 1 (N-[3-(3,4-dihydroxyphenyl)-1-oxo-2-propenyl]-2-hydroxybenzamide), 2E or 2Z or both, can be isolated from plants like Avena sativa, but not limited to them.

Therefore, any material or extract of these plants or any other plant material or extract containing the compound 1, preferably in an amount of at least 50 weight-%, more preferably in an amount of at least 70 weight-%, even more preferably in an amount of at least 90 weight-%, based on the total weight of the plant material or extract, is also encompassed by this expression. “Compound 1” means both “natural” (isolated) and “synthetic” (manufactured) compound 1. If extracts or parts of Avena sativa are used as alternative for compound 1 itself, these extracts or parts preferably do essentially not contain any one of the following components: tocopherols, tocols, flavonoids, non-flavonoid phenolic acids (such as avenanthramide, caffeic acid, ferulic acid).

The compound 2 (2-hydroxy-N-[1-oxo-3-(3,4,5-trimethoxyphenyl)-2-propenyl benzamide), 2E or 2Z or both, can be isolated as metabolite from plants like Alstonia lenormandii, but not limited to it.

Therefore, any material or extract of these plants or any other plant material or extract containing the compound 2, preferably in an amount of at least 1 weight-%, more preferably in an amount of at least 50 weight-%, even more preferably in an amount of at least 90 weight-%, based on the total weight of the plant material or extract, is also encompassed by this expression. “Compound 2” means both “natural” (isolated) and “synthetic” (manufactured) compound 2.

The compound 3 (3-(2,4-dimethoxyphenyl)-1-(2,5-dimethoxyphenyl)-2-propen-1-one), 2E or 2Z or both, can be isolated as minor metabolite from plants like Scutellaria indica, but not limited to them.

Therefore, any material or extract of these plants or any other plant material or extract containing the compound 3, preferably in an amount of at least 1 weight-%, more preferably in an amount of at least 50 weight-%, even more preferably in an amount of at least 90 weight-%, based on the total weight of the plant material or extract is also encompassed by this expression. “Compound 3” means both “natural” (isolated) and “synthetic” (manufactured) compound 3.

The compound 4, 2E or 2Z or both, can be isolated as trace metabolite from plants like Papaver pseudo orientale and the poppy plant, but not limited to them.

Therefore, any material or extract of these plants or any other plant material or extract containing the compound 4, preferably in an amount of at least 1 weight-%, more preferably in an amount of at least 50 weight-%, even more preferably in an amount of at least 90 weight-%, based on the total weight of the plant material or extract is also encompassed by this expression. “Compound 4” means both “natural” (isolated) and “synthetic” (manufactured) compound 4.

The compound 5 (3-phenyl-2-propenoic acid 3-[3-(4-methoxyphenyl)-3-oxo-1-propenyl]phenyl ester), 2E,1E or 2Z,1Z or both, can be isolated from cultivated callus cells of plants like Glycyrrhiza glabra (licorice), but not limited to them.

Therefore, any material or extract of these plants or any other plant material or extract containing the compound 5, preferably in an amount of at least 1 weight-%, more preferably in an amount of at least 50 weight-%, even more preferably in an amount of at least 90 weight-%, based on the total weight of the plant material or extract is also encompassed by this expression. “Compound 5” means both “natural” (isolated) and “synthetic” (manufactured) compound 5.

The compound 6 (3-(3,4-dimethoxyphenyl)-1-(4-methoxyphenyl)-2-propen-1-one), 2E or 2Z or both, can be isolated from plants like Glycyrrhiza glabra (licorice), but not limited to them.

Therefore, any material or extract of these plants or any other plant material or extract containing the compound 6, preferably in an amount of at least 1 weight-%, more preferably in an amount of at least 50 weight-%, even more preferably in an amount of at least 90 weight-%, based on the total weight of the plant material or extract is also encompassed by this expression. “Compound 6” means both “natural” (isolated) and “synthetic” (manufactured) compound 6.

The synthesis of compound 6 is e.g. described by Lin, Chun-Nan; Lee, Tai-Hua; Hsu, Mei-Feng; Wang, Jih-Pyang; Ko, Feng-Nien; Teng, Che-Ming in JPPMAB; J. Pharm. Pharmacol.; EN; 49; 5; 1997; 530-536, and by Patt, William C.; Edmunds, Jeremy J.; Repine, Joseph T.; Berryman, Kent A.; Reisdorph, Billy R.; et al. in JMCMAR; J. Med. Chem.; EN; 40; 7; 1997; 1063-1074.

The compound 7 (6-hydroxy-2-(4-methoxyphenyl)methylene]-3(2H)-benzofuranone) can be isolated from plants like Glycine max and Lygos raetam, but not limited to them.

Therefore, any material or extract of these plants or any other plant material or extract containing the compound 7, preferably in an amount of at least 1 weight-%, more preferably in an amount of at least 50 weight-%, even more preferably in an amount of at least 90 weight-%, based on the total weight of the plant material or extract is also encompassed by this expression. “Compound 7” means both “natural” (isolated) and “synthetic” (manufactured) compound 7.

The synthesis of compound 7 is e.g. described by Geissman and Harborne in JACSAT; J. Am. Chem. Soc.; 78; 1956; 832, 837.

The compound 8 (3-(2,4-dimethoxyphenyl)-1-(4-methoxyphenyl)-2-propen-1-one), 2E or 2Z or both, can be isolated from plants like Prunus cerasus, but not limited to them.

Therefore, any material or extract of these plants or any other plant material or extract containing the compound 8, preferably in an amount of at least 1 weight-%, more preferably in an amount of at least 50 weight-%, even more preferably in an amount of at least 90 weight-%, based on the total weight of the plant material or extract is also encompassed by this expression. “Compound 8” means both “natural” (isolated) and “synthetic” (manufactured) compound 8.

The synthesis of compound 8 is e.g. described by Kamat, Vinayak S.; Graden, David W.; Lynn, David G.; Steffens, John C.; Riopel, James L.; TELEAY in Tetrahedron Lett.; EN; 23; 15; 1982; 1541-1544.

The compound 9 can be isolated from plants like Primula officinalis and soybeans, but not limited to them.

Therefore, any material or extract of these plants or any other plant material or extract containing the compound 9, preferably in an amount of at least 1 weight-%, more preferably in an amount of at least 50 weight-%, even more preferably in an amount of at least 90 weight-%, based on the total weight of the plant material or extract is also encompassed by this expression. “Compound 9” means both “natural” (isolated) and “synthetic” (manufactured) compound 9.

The synthesis of compound 9 is e.g. described by Nagarathnam, Dhanapalan; Cushman, Mark, “A short and facile synthetic route to hydroxylated flavones. New syntheses of apigenin, tricin, and luteolin.” in Journal of Organic Chemistry 1991, 56(16), 4884-7.

The compound 10 (Diosmetin) can be isolated from from aerial parts of Valeriana spp., leaves of Digitalis spp., peel of lemon (Citrus limon), but not limited to them.

Therefore, any material or extract of these plants or any other plant material or extract containing the compound 10, preferably in an amount of at least 1 weight-%, more preferably in an amount of at least 50 weight-%, even more preferably in an amount of at least 90 weight-%, based on the total weight of the plant material or extract is also encompassed by this expression. “Compound 10” means both “natural” (isolated) and “synthetic” (manufactured) compound 10.

The synthesis of compound 10 is e.g. described by Teoule, R.; Chopin, J.; Mentzer, C., “A new synthesis of diosmetin and some of its derivatives.” in Bulletin de la Societe Chimique de France 1959, 854-5.

The compound 11 (4′,7-dimethoxyisoflavone) can be isolated from plants like Dalbergia violacea and Pterodon apparicioi heartwoods, but not limited to them.

Therefore, any material or extract of these plants or any other plant material or extract containing the compound 11, preferably in an amount of at least 1 weight-%, more preferably in an amount of at least 50 weight-%, even more preferably in an amount of at least 90 weight-%, based on the total weight of the plant material or extract is also encompassed by this expression. “Compound 11” means both “natural” (isolated) and “synthetic” (manufactured) compound 11.

The synthesis of compound 11 is e.g. described by Balasubramanian, Sreenivasan; Nair, Muraleedharan G., “An efficient “one pot” synthesis of isoflavones.” in Synthetic Communications 2000, 30(3), 469-484.

The compound 12 (Medicarpin) can be isolated from plants like Leguminosae subf. Papilionoideae, Osteophloeum platyspermum, Dalbergia spp., and Swartzia madagascariensis, but not limited to them.

Therefore, any material or extract of these plants or any other plant material or extract containing the compound 12, preferably in an amount of at least 1 weight-%, more preferably in an amount of at least 50 weight-%, even more preferably in an amount of at least 90 weight-%, based on the total weight of the plant material or extract is also encompassed by this expression. “Compound 12” means both “natural” (isolated) and “synthetic” (manufactured) compound 12.

The synthesis of compound 12 is e.g. described by Nabaei-Bidhendi, G.; Bannerjee, N. R.; “Convenient syntheses of 7-demethylhomopterocarpin and 7-demethylpterocarpin.” in Indian Journal of Chemistry, Section B: Organic Chemistry Including Medicinal Chemistry 1990, 29B(4), 366-8.

Beside the (pure) compounds 1 to 12 preferred are plant materials and plant extracts, especially those containing at least 10 weight-%, preferably at least 50 weight-%, more preferably at least 90 weight-%, of these compounds, based on the total weight of the plant material/extract.

The present invention is further directed to the use of a compound of the formula Ia/Ib/Ic/Id/Ie/If/Ig as defined above for the manufacture of a composition for the treatment of muscular disorders, the improvement of muscle function and muscle performance, disorders connected to impaired lipid metabolism, impaired glucose metabolism and impaired insulin action.

In preferred embodiments of the present invention this composition is used as physical performance enhancer, as endurance increaser, as muscle loss decreasing agent, as HDL cholesterol increaser, as triglyceride and cholesterol decreasing agent, as blood glucose controlling agent, as insulin sensitizer, as blood lipid lowering agent, as pancreatic β-cell function improver, as diabetes type 2 preventing agent and/or as Syndrome X preventing agent.

The present invention is also directed to a dietary composition containing at least a compound of the formula I or Ie as defined above, especially a dietary composition containing at least a compound of the formula Ia/Ib/Ic/Id/Ie/If/Ig

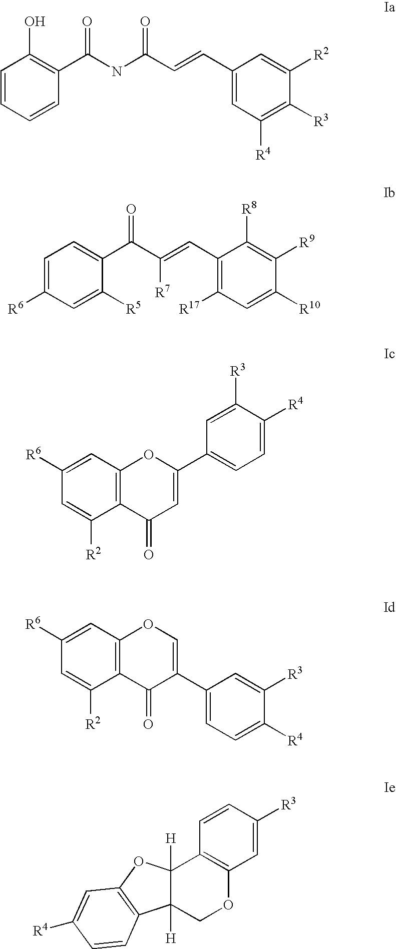

If see above, Ig see below

wherein

R is H, OH or C₁₋₆-alkyloxy; R³, R⁴ and R⁶ are independently from each other OH or C₁₋₆-alkyloxy;

R⁵ is H or C₁₋₆-alkyloxy; R⁷is H; or R⁵ and R⁷ are together —O—; R⁸ and R¹⁰ are independently from each other C₁₋₆-alkyloxy;

R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6.

R² is preferably hydrogen, hydroxyl or methoxy.

R³ and R⁴ are independently from each other preferably hydroxyl or methoxy.

R⁶ is preferably hydroxyl or methoxy.

R⁵ is preferably hydrogen or methoxy.

R⁷is preferably hydrogen.

R⁸ and R¹⁰ are preferably methoxy.

R⁹ is preferably hydrogen, methoxy or cinnamoyloxy.

Preferred is also a dietary composition containing a compound of the formula Ib, wherein R⁵ and R⁷ are together —O—.

Preferred is also a dietary composition containing a compound of the formula Ib, wherein R⁹ and R¹⁰ form together a group O—(CH₂)ₓ—O with x=1 or 2, especially wherein R⁹ and R¹⁰ form together the group O—CH₂—O.

R¹⁷ is preferably N-acetyl, N-methyl-2-aminoethyl.

In a preferred embodiment of the invention the dietary composition contains at least a compound selected from the group consisting of compounds 1 to 8 and 11, preferably consisting of compounds 1 to 6 and 11, more preferably consisting of compounds of the general formula If and the compound 11, even more preferably consisting of compounds 3, 4, 6 and 11, most preferably consisting of compounds 4 and 11, as defined above.

The term “dietary compositions” comprises any type of (fortified) food, (fortified) (animal) feed and beverages including also clinical nutrition, and also dietary supplements as well as the corresponding additives: food additives, beverage additives, feed additives. Also encompassed is functional food/feed i.e. a food/feed that has been enhanced with vitamins, other micronutrients or pharmaceuticals to provide further specific health benefits, as well as a nutraceutical, i.e. a pill or other pharmaceutical product that has nutritional value.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellyfying agents, gel forming agents, antioxidants and antimicrobials.

The present invention is also directed to a pharmaceutical composition containing at least one compound of the formula I or Ie or If, especially a pharmaceutical composition containing at least one compound of the formula Ia, Ib, Ic, Id or Ie or If, with the definitions of R² to R¹⁷ and the preferences as given above and a conventional pharmaceutical carrier.

Especially preferred is a pharmaceutical composition wherein the compound of the formulae Ia to Ie is selected from the group consisting of compounds 1 to 8 and 11, preferably consisting of compounds 1 to 6 and 11, more preferably consisting of compounds of the general formula If and the compound 11, even more preferably consisting of compounds 3, 4, 6 and 11, most preferably consisting of compounds 4 and 11, as defined above.

Beside a pharmaceutically acceptable carrier and at least one compound of the formulae I or Ie, especially of the formulae Ia to Ie with the definitions of R² to R¹⁷ and the preferences as given above or of the formula If as defined above or of the formula Ig as defined below, the pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The carrier material can be organic or inorganic inert carrier material suitable for oral/parenteral/injectable administration.

The dietary and pharmaceutical compositions according to the present invention may be in any galenic form that is suitable for administrating to the animal body including the human body, especially in any form that is conventional for oral administration, e.g. in solid form such as (additives/supplements for) food or feed, food or feed premix, fortified food or feed, tablets, pills, granules, dragées, capsules, and effervescent formulations such as powders and tablets, or in liquid form such as solutions, emulsions or suspensions as e.g. beverages, pastes and oily suspensions. The pastes may be filled into hard or soft shell capsules, whereby the capsules feature e.g. a matrix of (fish, swine, poultry, cow) gelatin, plant proteins or ligninsulfonate. Examples for other application forms are forms for transdermal, parenteral or injectable administration. The dietary and pharmaceutical compositions may be in the form of controlled (delayed) release formulations.

Examples for fortified food are cereal bars, bakery items such as cakes and cookies.

Beverages encompass non-alcoholic and alcoholic drinks as well as liquid preparations to be added to drinking water and liquid food. Non-alcoholic drinks are e.g. soft drinks, sport drinks, fruit juices, lemonades, teas and milk based drinks. Liquid food are e.g. soups and dairy products.

The compounds of the formulae I and Ie, especially the compounds of the formulae Ia to Ie with the definitions of R² to R¹⁷ and the preferences as given above or the compounds of the formula If as defined above or the compounds of the formula Ig as defined below as well as (mixtures of) plant materials and plant extracts containing them, preferably in an amount of at least 1 weight-% (except compound 1), more preferably in an amount of at least 50 weight-%, even more preferably in an amount of at least 90 weight-%, based on the total weight of the plant material or extract, and dietary/pharmaceutical compositions containing them are thus suitable for the treatment of mammals including humans.

The present invention is also directed to body care compositions containing at least one compound of the formula I or Ie, especially one compound of the formula Ia, Ib, Ic, Id or Ie with the definitions of R² to R¹⁷ and the preferences as given above or one compound of the formula If as defined above or one compound of the formula Ig as defined below and a conventional cosmetic carrier. Body care compositions encompass skin care preparations, preparations containing scents and/or fragrances, preparation, hair-care preparations, dentrifices, deodorant and antiperspirant, decorative preparations, light protection preparations and functional preparations, as well as preparations promoting/for improvement of skin wound healing and/or skin regeneration.

Examples of skin care preparations are, in particular, body oils, body lotions, body gels, treatment creams, skin protection ointments, shaving preparations, such as shaving foams or gels, skin powders, moisturizing gels, moisturizing sprays, revitalizing body sprays and peeling preparations.

Preparations containing scents and/or fragrances are in particular perfumes, toilet waters and shaving lotions (aftershave preparations).

Examples of hair care products are, for example, shampoo for humans and animals, hair conditioners, products for styling and treating hair, perming agents, hair sprays and lacquers, hair gels, hair fixatives and hair dying or bleaching agents.

Examples of dentifrices are in particular tooth cream, toothpastes, mouth-washes, mouth rinses, anti-plaque preparations and cleansing agents for dentures.

Examples of decorative preparations are in particular lipstick, nail varnishes, eye shadow, mascaras, dry and moist make-up, rouge, powders, depilatory agents, and suntan lotions.

Examples of functional preparations are cosmetic or dermatological compositions containing active ingredients such as hormone preparations, vitamin preparations, vegetable extract preparations and antibacterial preparations.

Body care products in accordance with the invention such as cosmetic and dermatological compositions can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse, foam or a spray foams, sprays, sticks or aerosols or wipes.

The body care products according to the invention can be in the form of a suspension or dispersion in solvents or fatty substances, or alternatively in the form of an emulsion or micro emulsion (in particular of O/W or W/O type, O/W/O or W/O/W-type), such as a cream or a milk, a vesicular dispersion, in the form of an ointment, a gel, a solid tube stick or an aerosol mousse. The emulsions can also contain anionic, nonionic, cationic or amphoteric surfactant.

The body care products or household products according to the invention can also contain usual adjuvants and additives, such as preservatives/antioxidants, fatty substances/oils, water, organic solvents, silicones, thickeners, softeners, emulsifiers, additional screening agents, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, light stabilizers, insect repellants, skin tanning agents, skin whitening agents, antibacterial agents, preservatives or any other ingredients usually formulated into cosmetics. The necessary amounts of the cosmetic and dermatological adjuvants and additives can, based on the desired product, easily be chosen by a skilled artisan in this field and will be illustrated in the examples, without being limited hereto.

Additional screening agents are advantageously selected from the compounds listed below without being limited thereto:

Examples of UV-B or broad spectrum screening agents, i.e. substances having absorption maximums between about 290 and 340 nm, which come into consideration for combination with the compounds of the present invention are for example the following organic and inorganic compounds:
Acrylates such as 2-ethylhexyl 2-cyano-3,3-diphenylacrylate (octocrylene, PARSOL® 340) and ethyl 2-cyano-3,3-diphenylacrylate;
Camphor derivatives such as 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor, and therephthalidene dicamphor sulfonic acid;
Cinnamate derivatives such as octyl methoxycinnamate (PARSOL® MCX), ethoxyethyl methoxycinnamate, diethanolamine methoxycinnamate (PARSOL® Hydro), and isoamyl methoxycinnamate, as well as cinnamic acid derivatives bond to siloxanes;
p-Aminobenzoic acid derivatives, such as p-aminobenzoic acid, 2-ethylhexyl p-dimethylaminobenzoate, N-oxypropylenated ethyl p-aminobenzoate and glyceryl p-aminobenzoate,
Benzophenones such as benzophenone-3, benzophenone-4,2,2′,4,4′-tetrahydroxy-benzophenone and 2,2′-dihydroxy-4,4′-dimethoxybenzophenone;
Esters of Benzalmalonic acid such as di-(2-ethylhexyl)4-methoxybenzalmalonate;
Esters of 2-(4-ethoxy-anilinomethylene)propandioic acid such as 2-(4-ethoxy anilinomethylene)propandioic acid diethyl ester as described in EP-A 0 895 776;
Organosiloxane compounds containing benzmalonate groups as described in EP-A 0 358 584, EP-A 0 538 431 and EP-A 0 709 080;
Drometrizole trisiloxane (Mexoryl XL);
Pigments such as microparticulated TiO2, and the like. The term “microparticulated” refers to a particle size from about 5 nm to about 200 nm, particularly from about 15 nm to about I 00 nm. The TiO2 particles may also be coated by metal oxides such as e.g. aluminum or zirconium oxides or by organic coatings such as e.g. polyols, methicone, aluminum stearate, alkyl silane. Such coatings are well known in the art.
Imidazole derivatives such as e.g. 2-phenyl benzimidazole sulfonic acid and its salts (PARSOL® HS). Salts of 2-phenyl benzimidazole sulfonic acid are e.g. alkali salts such as sodium- or potassium salts, ammonium salts, morpholine salts, salts of primary, sec. and tert. amines like monoethanolamine salts and diethanolamine salts.
Salicylate derivatives such as isopropylbenzyl salicylate, benzyl salicylate, butyl salicylate, octyl salicylate (NEO HELIOPAN OS), isooctyl salicylate or homomenthyl salicylate (homosalate, HELIOPAN).
Triazine derivatives such as octyl triazone (UVINUL T-150), dioctyl butamido triazone (UVASORB HEB) and bis ethoxyphenol methoxyphenyl triazine (Tinosorb S).

Examples of broad spectrum or UV A screening agents i.e. substances having absorption maximums between about 320 and 400 nm, which come into consideration for combination with the compounds of the present invention are for example the following organic and inorganic compounds:
Dibenzoylmethane derivatives such as 4-tert. butyl-4′-methoxydibenzoyl-methane (PARSOL® 1789), dimethoxydibenzoylmethane and isopropyldibenzoylmethane;
Benzotriazole derivatives such as 2,2′-methylene-bis-(6-(2H-benzotriazole-2-yl)-4-(1,1,3,3,-tetramethylbutyl)-phenol (TINOSORB M);
Phenylene-1,4-bis-benzimidazolsulfonic acids or salts such as 2,2-(1,4-phenylene)bis-(1H-benzimidazol-4,6-disulfonic acid) (Neoheliopan AP);
Amino substituted hydroxybenzophenones such as 2-(4-Diethylamino-2-hydroxy-benzoyl)-benzoic acid hexylester (Uvinul A plus) as described in EP-A 1 046 391;
Pigments such as microparticulated ZnO or TiO2. The term “microparticulated” refers to a particle size from about 5 nm to about 200 nm, particularly from about 15 nm to about 100 nm. The particles may also be coated by other metal oxides such as e.g. aluminum or zirconium oxides or by organic coatings such as e.g. polyols, methicone, aluminum stearate, alkyl silane. Such coatings are well known in the art.

As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g.,
3,3-Diphenylacrylate derivatives as described in EP-A 0 514 491 and EP-A 0 780 119;
Benzylidene camphor derivatives as described in U.S. Pat. No. 5,605,680;
Organosiloxanes containing benzmalonate groups as described in EP-A 0 358 584, EP-A 0 538 431 and EP-A 0 70 9080.

Based on the invention all known antioxidants usually formulated into body care, household and fragrance products can be used. Especially preferred are antioxidants chosen from the group consisting of amino acids (e.g. glycine, histidine, tyrosine, tryptophan) and their derivatives, imidazole (e.g. urocanic acid) and derivatives, peptides such as D,L-carnosine, D-carnosine, L-carnosine and derivatives (e.g. anserine), carotenoids, carotenes (e.g. α-carotene, β-carotene, lycopene) and derivatives, chlorogenic acid and derivatives, lipoic acid and derivatives (e.g. dihydrolipoic acid), aurothioglucose, propylthiouracil and other thiols (e.g. thioredoxine, glutathione, cysteine, cystine, cystamine and its glycosyl-, N-acetyl-, methyl-, ethyl-, propyl-, amyl-, butyl- and lauryl-, palmitoyl-; oleyl-, y-linoleyl-, cholesteryl- and glycerylester) and the salts thereof, dilaurylthiodipropionate, distearylthiodipropionate, thiodipropionic acid and its derivatives (ester, ether, peptides, lipids, nucleotides, nucleosides and salts) as well as sulfoximine compounds (such as buthioninsulfoximine, homocysteinesulfoximine, buthioninsulfone, penta-, hexa-, heptathioninsulfoximine) in very low compatible doses (e.g. pmol bis μmol/kg), additionally (metal)-chelators (such as α-hydroxyfatty acids, palmic-, phytinic acid, lactoferrin), β-hydroxyacids (such as citric acid, lactic acid, malic acid), huminic acid, gallic acid, gallic extracts, bilirubin, biliverdin, EDTA, EGTA and its derivatives, unsaturated fatty acids and their derivatives (such as γ-linoleic acid, linolic acid, oleic acid), folic acid and its derivatives, ubiquinone and ubiquinol and their derivatives, vitamin C and derivatives (such as ascorbylpalmitate and ascorbyltetraisopalmitate, Mg-ascorbylphosphate, Na-ascorbylphosphate, ascorbyl-acetate), tocopherol and derivates (such as vitamin-E-acetate), mixtures of nat. vitamin E, vitamin A and derivatives (vitamin-A-palmitate and -acetate) as well as coniferylbenzoate, rutinic acid and derivatives, α-glycosylrutin, ferulic acid, furfurylideneglucitol, carnosine, butyl-hydroxytoluene, butylhydroxyanisole, trihydroxybutyrophenone, urea and its derivatives, mannose and derivatives, zinc and derivatives (e.g. ZnO, ZnSO4), selen and derivatives (e.g. selenomethionin), stilbenes and derivatives (such as stilbenoxide, trans-stilbenoxide) and suitable derivatives (salts, esters, ethers, sugars, nucleotides, nucleosides, peptides and lipids) of the named active ingredients.

One or more preservatives/antioxidants may be present in an amount of at least 0.01 wt. % of the total weight of the composition. Preferably about 0.01 wt. % to about 10 wt. % of the total weight of the composition of the present invention is present. Most preferred, one or more preservatives/antioxidants are present in an amount about 0.1 wt. % to about 1 wt. %.

Typically formulations also contain surface active ingredients like emulsifiers, solubilizers and the like. An emulsifier enables two or more immiscible components to be combined homogeneously. Moreover, the emulsifier acts to stabilize the composition. Emulsifiers that may be used in the present invention in order to form O/W, W/O, O/W/O or W/O/W emulsions/microemulsions include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polyglyceryl-4 oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof. Further exemplary emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glycerides phosphate and mixtures thereof. Furthermore, one or more synthetic polymers may be used as an emulsifier. For example, PVP eicosene copolymer, acrylates/C10-30 alkyl acrylate crosspolymer, acrylates/steareth-20 methacrylate copolymer, PEG-22/dodecyl glycol copolymer, PEG-45/dodecyl glycol copolymer, and mixtures thereof. The preferred emulsifiers are cetyl phosphate (Amphisol® A), diethanolamine cetyl phosphate (Amphisol®), potassium cetyl phosphate (Amphisol® K), PVP Eicosene copolymer, acrylates/C10-30-alkyl acrylate crosspolymer, PEG-20 sorbitan isostearate, sorbitan isostearate, and mixtures thereof. The one or more emulsifiers are present in a total amount of at least 0.01 wt. % of the total weight of the composition. Preferably about 0.01 wt. % to about 20 wt. % of the total weight of the composition of the present invention is used. Most preferred, about 0.1 wt. % to about 10 wt. % of emulsifiers are used.

The lipid phase can advantageously be chosen from:
mineral oils and mineral waxes;
oils such as triglycerides of caprinic acid or caprylic acid, preferable castor oil;
oils or waxes and other natural or synthetic oils, in an preferred embodiment esters of fatty acids with alcohols e.g. isopropanol, propyleneglycol, glycerin or esters of fatty alcohols with carbonic acids or fatty acids;
alkylbenzoates; and/or
silicone oils such as dimethylpolysiloxane, diethylpolysiloxane, diphenylpolysiloxane, cyclomethicones and mixtures thereof.

Exemplary fatty substances which can be incorporated in the oil phase of the emulsion, microemulsion, oleo gel, hydrodispersion or lipodispersion of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropylmyristate, isopropylpalmitate, isopropylstearate, isopropyloleate, n-butylstearate, n-hexyllaureate, n-decyloleate, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components suitable for use in the formulation of the present invention include polar oils such as lecithins and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicone (octamethylcyclotetrasiloxane; cetyldimethicone, hexamethylcyclotrisiloxane, polydimethylsiloxane, poly(methylphenylsiloxane) and mixtures thereof.

Other fatty components which can advantageously be incorporated in formulations of the present invention are isoeikosane; neopentylglycoldiheptanoate; propyleneglycoldicaprylate/dicaprate; caprylic/capric/diglycerylsuccinate; butyleneglycol caprylat/caprat; C12-13-alkyllactate; di-C12-13-alkyltartrate; triisostearin; dipentaerythrityl hexa-caprylat/hexacaprate; propyleneglycolmonoisostearate; tricaprylin; dimethylisosorbid. Especially beneficial is the use of mixtures C12-1 5-alkylbenzoate and 2-ethylhexylisostearate, mixtures C12-15-alkylbenzoate and isotridecylisononanoate as well as mixtures of C12-15-alkylbenzoate, 2-ethylhexylisostearate and isotridecylisononanoate.

The oily phase of the formulation of the present invention can also contain natural vegetable or animal waxes such as bee wax, china wax, bumblebee wax and other waxes of insects as well as shea butter and cocoa butter.

A moisturizing agent may be incorporated into a product of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, capric/caprylic triglyceraldehydes, cholesterol, silicones such as dimeticone, cyclometicone, almond oil, jojoba oil, avocado oil, castor oil, sesame oil, sunflower oil, coconut oil and grape seed oil, cocoa butter, olive oil aloe extracts, fatty acids such as oleic and stearic, fatty alcohols such as cetyl and hexadecyl (ENJAY), diisopropyl adipate, hydroxybenzoate esters, benzoic acid esters of C9-15-alcohols, isononyl iso-nonanoate, ethers such as polyoxypropylene butyl ethers and polyoxypropylene cetyl ethers, and C12-15-alkyl benzoates, and mixtures thereof. The most preferred emollients are hydroxybenzoate esters, aloe vera, C12-15-alkyl benzoates, and mixtures thereof.

An emollient is present in an amount of about 1 wt. % to about 20 wt. % of the total weight of the product. The preferred amount of emollient is about 2 wt. % to about 15 wt. %, and most preferably about 4 wt. % to about 10 wt. %.

Moisturizers that bind water, thereby retaining it on the skin surface are called humectants. Examples of humectants which can be incorporated into a product of the present invention are glycerin, polypropylene glycol, polyethylene glycol, lactic acid, pyrrolidone carboxylic acid, urea, phospholipids, collagen, elastin, ceramides, lecithin sorbitol, PEG-4, and mixtures thereof. Additional suitable moisturizers are polymeric moisturizers of the family of water soluble and/or swellable/and/or with water gelating polysaccharides such as hyaluronic acid, chitosan and/or a fucose rich polysaccharide which is e.g. available as Fucogel® 1000 (CAS-Nr. 178463-23-5) by SOLABIA S.

One or more humectants are optionally present at about 0.5 wt. % to about 8 wt. % in a product of the present invention, preferably about 1 wt. % to about 5 wt. %.

The aqueous phase of the products of the present invention can contain the usual cosmetic additives such as alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or-monobutylether, diethyleneglycol monomethyl- or monoethylether and analogue products, polymers, foam stabilizers; electrolytes and especially one or more thickeners. Thickeners that may be used in formulations of the present invention to assist in making the consistency of a product suitable include carbomer, siliciumdioxide, magnesium and/or aluminum silicates, beeswax, stearic acid, stearyl alcohol polysaccharides and their derivatives such as xanthan gum, hydroxypropyl cellulose, polyacrylamides, acrylate crosspolymers preferably a carbomer, such as carbopole® of type 980, 981, 1382, 2984, 5984 alone or mixtures thereof. Examples of neutralizing agents which may be included in the product of the present invention to neutralize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing.

The neutralizing agent can be present in an amount of about 0.01 wt. % to about 8 wt. % in the product of the present invention, preferably, 1 wt. % to about 5 wt. %.

The addition of electrolytes into the product of the present invention may be necessary to change the behavior of a hydrophobic emulsifier. Thus, the emulsions/microemulsions of this invention may contain preferably electrolytes of one or several salts including anions such as chloride, sulfates, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkylammonium, alkali- or alkaline earth metals, magnesium-, iron- or zinc-ions are selected. Especially preferred salts are potassium and sodium chloride, magnesium sulfate, zinc sulfate and mixtures thereof.

Electrolytes can be present in an amount of about 0.01 wt. % to about 8 wt. % in the product of the present invention.

The addition of further light stabilizers may be desirable. Such light stabilizers are e.g. known as sterically hindered amine light stabilizer (HALS) which can be of monomeric or polymeric nature. They are for example selected from the group consisting of N,N′-bisformyl-N,N′-bis-(2,2,6,6-tetramethyl-4-piperidinyl)-hexamethylenediamine (Uvinul 4050 H), bis-(2,2,6,6-tetramethyl-4-piperidyl)sebacate (Uvinul 4077 H), (bis-(1,2,2,6,6-pentamethyl-4-piperidyl)-sebacate+methyl-(1,2,2,6,6-pentamethyl-4-piperidyl)sebacate. (Uvinul 4092 H), bis (2,2,6,6-tetramethylpiperidin-4-yl) sebacate, bis (2,2,6,6-tetramethyl-piperidin-4-yl)succinate, bis(1,2,2,6,6-pentamethylpiperidin-4-yl)sebacate, n-butyl-3,5-di-tert-butyl-4-hydroxybenzyl-malonic acid bis(1,2,2,6,6-pentamethylpiperidyl) ester, the condensate of 1-hydroxyethyl-2,2,6,6-tetramethyl-4-hydroxypiperidine and succinic acid, the condensate of N,N′-bis(2,2,6,6-tetramethyl-4-piperidyl)hexamethylenediamine and 4-tert-octylamino-2,6-dichloro-1,3,5-s-triazine, tris(2,2,6,6-tetramethyl-4-piperidyl)nitrilotriacetate, tetrakis(2,2,6,6-tetra-methyl-4-piperidyl)-1,2,3,4-butane-tetranoate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-penta-methylpiperidyl)-2-n-butyl-2-(2-hydroxy-3,5-di-tert-butylbenzyl)malonate, 3-n-octyl-7,7,9,9-tetramethyl-1,3,8-triazaspiro[4.5]decan-2,4-dione, the condensate of N,N-bis(2,2,6,6-tetramethyl-4-piperidyl)hexamethylenediamine and 4-morpholino-2,6-dichloro-1,3,5-triazine, the condensate of 2-chloro-4,6-di(4-n-butylamino-2,2,6,6-tetra-methylpiperidyl)-1,3,5-triazine and 1,2-bis(3-aminopropylamino)ethane, the condensate of 2-chloro-4,6-di(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4.5]-decane-2,4-dione, 3-dodecyl-1-(2,2,6,6-tetramethyl-4-piperidyl)pyrrolidin-2,5-dione, 3-dodecyl-1-(1,2,2,6,6-pentamethy-14-piperidyl)-pyrrolidine-2,5-dione, a mixture of 4-hexadecyloxy-and 4-stearyloxy-2,2,6,6-tetramethylpiperidine, the condensate of N,N′-bis(2,2,6,6-tetramethyl-4-piperidyl)hexamethylenediamine and 4-cyclohexylamino-2,6-dichloro-1,3,5-triazine, the condensate of 1,2-bis(3-aminopropyl-amino)ethane and 2,4,6-trichloro-1,3,5-triazine and 4-butylamino-2,2,6,6-tetramethyl-piperidine (CAS reg. No. [136504-96-6]); (2,2,6,6-tetramethyl-4-piperidyl)-n-dodecyl-succinimide, (1,2,2,6,6-pentamethyl-4-piperidyl)-n-dodecylsuccinimide, 2-undecyl-7,7,9,9-tetramethyl-1-oxa-3,8-diaza-4-oxo-spiro[4,5]decane, the reaction product of 7,7,9,9-tetramethyl-2-cycloundecyl-1-oxa-3,8-diaza-4-oxospiro[4,5]decane and epichlorohydrin without being limited thereto.

Examples of insect repellants which can be used in body care products according to the invention are for example N,N-diethyl-m-toluamide, 1,2-pentanediol or insect repellant 3535.

Examples of self tanning ingredients are e.g. dihydroxyacetone and/or erythrulose or dihydroxy acetone and/or dihydroxyacetone precursors as described in WO 01/85124 and/or erythrulose.

Examples of skin whitening ingredients are for example vitamin C, sodium ascorbyl phosphate and magnesium ascorbyl phosphate.

Examples of deodorizing active ingredients which come into consideration are anti-perspirants such as aluminum chlorohydrates, aluminum hydroxyacetates and acidic aluminum/zirconium salts. Esterase inhibitors may be added as further deodorizing active ingredients. Such inhibitors are preferably trialkyl citrates, such as trimethyl citrate, tri-propyl citrate, triisopropyl citrate, tributyl citrate and especially triethyl citrate (Hydagen CAT, Henkel), which inhibit enzyme activity and hence reduce odor formation. Further substances that come into consideration as esterase inhibitors are sterol sulfates or phosphates, for example lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester and hydroxy-carboxylic acids and esters thereof, for example citric acid, malic acid, tartaric acid or tartaric acid diethyl ester. Antibacterial active ingredients that influence the germ flora and kill or inhibit the growth of sweat-decomposing bacteria can likewise be present in the preparations (especially in stick preparations). Other antibacterials which could be present are chitosan, phenoxyethanol and chlorhexidinegluconate-5-chloro-2-(2,4-dichloro-phenoxy)-phenol (Triclosan, Irgasan, Ciba Specialty Chemicals Inc.).

Examples of anti-dandruff agents which may be used are dimbazole, octopirox and zinc pyrithione. Customary film formers include, for example, chitosan, microcrystalline chitosan, quaternised chitosan, polyvinylpyrrolidone, vinylpyrrolidone/vinyl acetate copolymers, polymers of quaternary cellulose derivatives containing a high proportion of acrylic acid, collagen, hyaluronic acid and salts thereof and similar compounds.

Examples of preservatives include Methyl-, Ethyl-, Propyl-, Butylparabens, Benzalkonium chloride, 2-Bromo-2-nitro-propane-1,3-diol, Dehydroacetic acid, Diazolidinyl Urea, 2-Dichlorobenzyl alcohol, DMDM hydantoin, Formaldehyde solution, Methyidibromoglutaronitrile, Phenoxyethanol, Sodium Hydroxymethylglycinate, Imidazolidinyl Urea, Triclosan and further substance classes listed in the following reference: K. F. De Polo-A short textbook of cosmetology, Chapter 7, Table 7-2,7-3, 7-4 and 7-5, p 210-219.

Typical examples of bacteria-inhibiting agents are preservatives that have a specific action against gram-positive bacteria, such as 2,4,4′-trichloro-2′-hydroxydiphenyl ether, chlorhexi-dine (1,6-di(4-chlorophenyl-biguanido)hexane)or TCC (3,4,4′-trichloro-carbanilide). A large number of aromatic substances and ethereal oils also have antimicrobial properties. Typical examples are the active ingredients eugenol, menthol and thymol in clove oil, mint oil and thyme oil. A natural deodorizing agent of interest is the terpene alcohol farnesol (3,7,11-tri-methyl-2,6,10-dodecatrien-1-ol), which is present in lime blossom oil. Glycerolmonolaurate has also proved to be a bacteriostatic agent.

The amount of the additional bacteria-inhibiting agents present is usually from 0.1 to 2 wt. %, based on the solids content of the preparations.

The present stabilizer composition is especially suitable for stabilizing body care products, in particular:
skin-care preparations, e. g. skin-washing and cleansing preparations in the form of tablet-form or liquid soaps, soapless detergents or washing pastes,
bath preparations, e. g. liquid (foam baths, milks, shower preparations) or solid bath preparations, e. g. bath cubes and bath salts;
skin-care preparations, e. g. skin emulsions, multi-emulsions or skin oils; body oils, body lotions, body gels; skin protection ointments;
cosmetic personal care preparations, e. g. facial make-up in the form of day creams or powder creams, face powder (loose or pressed), rouge or cream make-up, eye-care preparations, e. g. eye shadow preparations, mascara, eyeliner, eye creams or eye-fix creams; lip-care preparations, e. g. lipsticks, lip gloss, lip contour pencils, nail-care preparations, such as nail varnish, nail varnish removers, nail hardeners or cuticle removers;
foot-care preparations, e. g. foot baths, foot powders, foot creams or foot balsams, special deodorants and antiperspirants or callus-removing preparations;
light-protective preparations, such as sun milks, lotions, creams or oils, unblocks or tropicals, pre-tanning preparations or after-sun preparations;
skin-tanning preparations, e. g. self-tanning creams;
depigmenting preparations, e. g. preparations for bleaching the skin or skin-lightening preparations;
insect-repellents, e. g. insect-repellent oils, lotions, sprays or sticks;
deodorants, such as deodorant sprays, pump-action sprays, deodorant gels, sticks or roll-ons;
antiperspirants, e. g. antiperspirant sticks, creams or roll-ons;
preparations for cleansing and caring for blemished skin, e. g. synthetic detergents (solid or liquid), peeling or scrub preparations or peeling masks;
hair-removal preparations in chemical form (depilation), e. g. hair-removing powders, liquid hair-removing preparations, cream-or paste-form hair-removing preparations, hair-removing preparations in gel form or aerosol foams;
shaving preparations, e. g. shaving soap, foaming shaving creams, non-foaming shaving creams, foams and gels, pre-shave preparations for dry shaving, aftershaves or aftershave lotions;
scent or fragrance preparations, e. g. scent, fragrance and/or odorant ingredient containing preparations such as perfumes, eau de Colognes, eau de toilettes, eau de perfumes, eau de toilettes, perfume oils or perfume creams;
cosmetic hair-treatment preparations, e. g. hair-washing preparations in the form of shampoos and conditioners, hair-care preparations, e. g. pre-treatment preparations, hair tonics, styling creams, styling gels, pomades, hair rinses, treatment packs, intensive hair treatments, hair-structuring preparations, e. g. hair-waving preparations for permanent waves (hot wave, mild wave, cold wave), hair-straightening preparations, liquid hair-setting preparations, hair foams, hairsprays, bleaching preparations, e. g. hydrogen per-oxide solutions, lightening shampoos, bleaching creams, bleaching powders, bleaching pastes or oils, temporary, semi-permanent or permanent hair colorants, preparations containing self-oxidizing dyes, or natural hair colorants, such as henna or chamomile;
dentifrices, in particular tooth creams, toothpastes, mouth-washes, mouth rinses, anti-plaque preparations and cleansing agents for dentures;
decorative preparations, in particular lipsticks, nail varnishes, eye shadows, mascaras, dry and moist make-up, rouge, powders, depilatory agents and suntan lotions;
cosmetic formulations containing active ingredients, in particular hormone preparations, vitamin preparations, vegetable extract preparations and antibacterial preparations.

The final formulations listed may exist in a wide variety of presentation forms, for example in the form of liquid preparations, as a W/O, O/W, O/W/O, W/O/W or PIT emulsion and all kinds of micro emulsions, in the form of a gel,-in the form of an oil, a cream, milk or lotion, in the form of a stick, in the form of a spray (spray with propellant gas or pump-action spray) or an aerosol,-in the form of a foam, or in the form of a paste.

Of special importance as cosmetic preparations for the skin according to the invention are colorant, dye, active ingredient, scent, fragrance or mixtures thereof containing preparations, such as sun milks, lotions, creams, wipes, oils, sun blocks or tropicals, pre-tanning preparations or after-sun preparations, also skin-tanning preparations, for example self-tanning creams.

Of special importance as cosmetic preparations for the hair are the above-mentioned preparations for hair treatment, especially hair-washing preparations in the form of shampoos, hair conditioners, hair-care preparations, e. g. pre-treatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

The compounds of the formulae I and Ie, especially the compounds of the formulae Ia to Ig with the definitions of the substituents and the preferences as given above and below as well as (mixtures of) plant materials and plant extracts containing them, preferably in an amount of at least 1 weight-%, more preferably in an amount of at least 50 weight-%, even more preferably in an amount of at least 90 weight-%, based on the total weight of the plant material or extract, and body care compositions containing them are thus suitable for the topical treatment of mammals including humans.

Therefore, the invention relates to a method for the treatment of a disorder connected to impaired glucose metabolism and impaired insulin action in mammals including humans, said method comprising administering an effective dose of a compound of the formula I or Ie, especially of a compound of the formula Ia, Ib, Ic, Id or Ie or If or Ig, as defined herein to mammals including humans which are in need thereof.

Mammals in the context of the present invention include humans. Preferred “mammals” are humans, and pets such as cats, dogs, horses, dromedaries, and elephants, especially dogs.

In the context of this invention “treatment” also encompasses co-treatment as well as control and or prevention. In the context of this invention the term “disorder” also encompasses diseases. Furthermore, “treatment” also encompasses the use by healthy individuals, who seek for better fitness, body shape, or skin appearance. “Prevention” in the context of the present invention encompasses also the reduction of risk of getting a certain disorder/disease as mentioned herein or reducing the incidence of getting a certain disorder/disease as mentioned herein.

For humans a suitable daily dosage of a compound of the formula I or Ie, especially of the formulae Ia to Ie with the definitions of R² to R¹⁷ and the preferences as given above or of a compound of formula If as defined above or of a compound of formula Ig as defined below, for the purposes of the present invention may be within the range from 0.01 mg per kg body weight to 50 mg per kg body weight per day, i.e. 0.7 mg-3500 mg for a 70 kg person. More preferred is a daily dosage of 0.1 to 25 mg per kg body weight (i.e. 7 mg-1750 mg for a 70 kg person), and especially preferred is a daily dosage of 0.3 to 15 mg per kg body weight, i.e. 21 mg-1050 mg for a 70 kg person. The amount of a plant material or plant extract containing such compound of the formulae Ia to If can be calculated accordingly.

In solid dosage unit preparations for humans, the compound of the formula I or Ie, especially of the formulae Ia to Ie with the definitions of R² to R¹⁷ and the preferences as given above or of a compound of formula If as defined above or of a compound of formula Ig as defined below is suitably present in an amount from 0.25 mg to 1000 mg, preferably from 2 mg to 200 mg per dosage unit.

In dietary compositions, especially in food and beverages for humans, the compound of the formula I or Ie, especially of the formulae Ia to Ie with the definitions of R² to R¹⁷ and the preferences as given above or of a compound of formula If as defined above or of a compound of formula Ig as defined below, may suitably be present in an amount of from 7 mg to 1750 mg, preferably, from 20 mg to 1000 mg per serving (serving size can be e.g. 500 mg for a lozenge, 50 g for bread or 250 mL for a beverage).

In food and drinks in a preferred embodiment of the invention the amount of the compound of the formula I or Ie, especially of the formulae Ia to Ie with the definitions of R² to R¹⁷ and the preferences as given above or of a compound of formula If as defined above or of a compound of formula Ig as defined below, may be 20 mg to 1000 mg per serving.

For dogs a suitable daily dosage of a compound of the formula I or Ie, especially of the formulae Ia to Ie with the definitions of R² to R¹⁷ and the preferences as given above or of a compound of formula If as defined above or of a compound of formula Ig as defined below, for the purposes of the present invention may be within the range from 0.04 mg per kg body weight to 500 mg per kg body weight per day. More preferred is a daily dosage of 0.4 mg to 100 mg per kg body weight, and especially preferred is a daily dosage of 1 mg to 50 mg per kg body weight.

The present invention is also directed to the use of compounds of the general formulae I and Ie as defined above, especially to the use of compounds of the general formulae Ia to Ie or of a compound of formula If as defined above or of a compound of formula Ig as defined below,

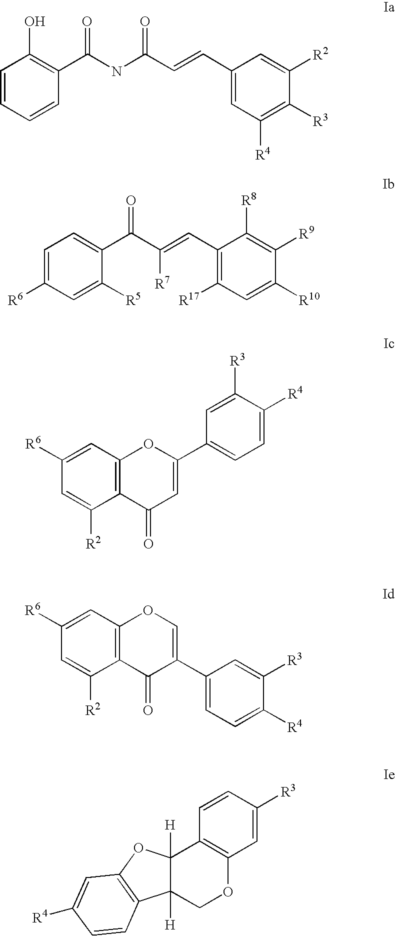

wherein

R² is H, OH or C₁₋₆-alkyloxy (preferably methoxy); R³, R⁴ and R⁶ are independently from each other OH or C₁₋₆-alkyloxy (preferably methoxy);

R⁵ is H or C₁₋₆-alkyloxy (preferably H or methoxy, more preferably methoxy); R⁷ is H; or

R⁵ and R⁷ are together —O—; R⁸ and R¹⁰ are independently from each other C₁₋₆-alkyloxy (preferably methoxy);

R⁹ is H, C₁₋₆-alkyloxy (preferably methoxy) or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6 (preferably (N-acyl,N-methyl)-2-aminoethyl), as medicament.

### Most Preferred Embodiment of the Present Invention

The most preferred embodiment of the present invention is directed to the use of a compound of the general formula Ig

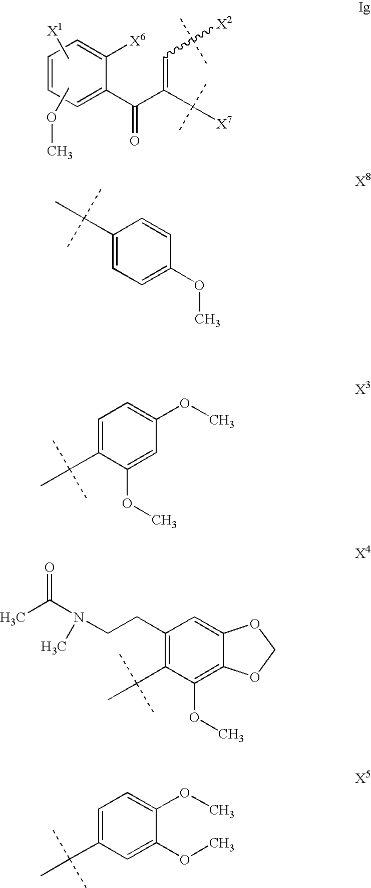

with X¹ being H or CH₃, and

X² being X³, X⁴ or X⁵; or X² and X⁶ together forming an oxygen bond

(i.e. —X⁶—X²—═—O—); and

X⁷being H or X⁸,

with the proviso that X⁶═X¹ and X⁷═H if X²═X³, X⁴ or X⁵; and

with the further proviso that X¹═H and X⁷═X⁸ if X² and X⁶ together form an oxygen bond (i.e. —X⁶—X²—═—O—),

for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans; as well as to the use of such a compound of the general formula Ig for the manufacture of a composition (dietary, bodycare or pharmaceutical composition as defined and with the preferences as given above) for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

Especially preferred for said use are compounds 3, 4, 6 and 11 (see FIG. 2 and FIG. 4) which fall under general formula Ig. More preferred are compounds 4 and 11, most preferred is compound 11.

From the uses listed the following ones are especially preferred:

enhancing the muscle function in mammals including humans,

enhancing the endurance of mammals including humans,

improving the body shape of mammals including humans and

improving the muscle:fat ratio in mammals including humans.

The present invention is further most preferred directed to a compound of the general formula Ig

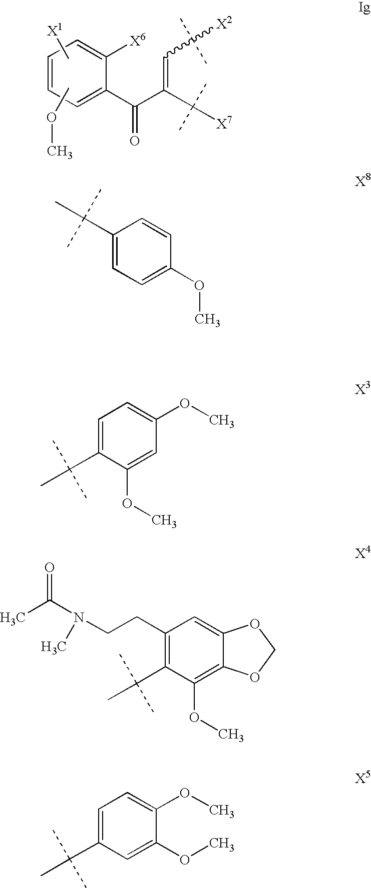

with X¹ being H or CH₃, and

X² being X³, X⁴ or X⁵; or X² and X⁶ together forming an oxygen bond

(i.e. —X⁶—X²—═—O—); and

X⁷ being H or X⁸,

with the proviso that X⁶═X¹ and X⁷═H if X²═X³, X⁴ or X⁵; and

with the further proviso that X¹═H and X⁷═X⁸ if X² and X⁶ together form an oxygen bond (i.e. —X⁶—X²—═—O—),

for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

A further most preferred embodiment of the present invention are compositions, especially dietary, body care or pharmaceutical compositions, as defined in present claims 4 and 5, the use of such composition according to claim 6 and methods for using such compounds of general formula Ig with the preferences as given above or compositions containing them as defined in the context of the present invention according to claims 7 and 8.

The invention is illustrated further by the following examples.

### EXAMPLES

### Example 1

### Soft Gelatin Capsule

Soft gelatin capsules are prepared by conventional procedures providing a dose of a compound of the formula I or Ie of 200 mg. A suitable daily dose is 1 to 5 capsules. Other ingredients: glycerol, water, gelatine, vegetable oil. When administered in that dosage for a period of two months to a man or woman at the age of 60 to 75, the walking distance for such a person may be increased by 10%.

### Example 2

### Hard Gelatin Capsule

Hard gelatin capsules are prepared by conventional procedures providing a dose of a compound of the formula I or Ie of 100 mg. A suitable daily dose is 1 to 5 capsules. When administered in that dosage for a period of two months to a man or woman at the age of 30 to 40, the running distance for such a person may be increased by 5%.

Other ingredients:

Fillers: lactose or cellulose or cellulose derivatives q.s.

Lubricant: magnesium stearate if necessary (0.5%)

### Example 3

### Tablet

Tablets are prepared by conventional procedures providing as active ingredient 50 mg of a compound of the formula I or Ie per tablet, and as excipients microcrystalline cellulose, silicone dioxide (SiO₂), magnesium stearate, crospovidone NF (which is a disintegration agent) ad 500 mg.

### Example 4

### Orange Juice Drink coloured with 30 mg β-Carotene 10% CWS

| Ingredients | [g] |
|---|---|
| Sugar syrup 64° Brix | 156.2 |
| Sodium benzoate | 0.2 |
| Ascorbic acid, fine powder | 0.2 |
| Citric acid 50% w/w | 5.0 |
| Pectin solution 2% w/w | 10.0 |
| compound of the formula I or Ie | 0.5 |
| Juice compound* | 30.0 |
| Water to | 250.0 |

Preparation

Dissolve sodium benzoate in water whilst stirring;

Continue stirring and add sugar syrup, ascorbic acid, citric acid, pectin solution, juice compound, one after the other. Do not use a high speed mixer;

Dilute the bottling syrup with (carbonated) water to one liter of beverage.

| *Ingredients Juice compound | [g] |
|---|---|
| Orange juice concentrate 65° Brix | 483.3 |
| Lemon Juice Concentrate 45° Brix | 173.3 |
| Oily orange flavour | 5.0 |
| β-Carotene 10% CWS as 10% stocksolution | 10.0 |
| Deionized water | 328.4 |

Preparation of Juice Compound

Add the deionized water to the juice concentrates, stir gently and allow the juice concentrates to hydrate.

Add the oily flavour and β-Carotene 10% CWS stocksolution and pre-emulsify in a rotor-stator-homogenizer.

Homogenize in a high-pressure homogenizer at 200 bar.

Addition of β-Carotene 10% CWS

β-Carotene 10% CWS should be added to the juice compound as a 1-10% stocksolution in deionized water.

The orange juice drink contains 3 ppm β-carotene.

### Example 5

### Influence of 4′,7-dimethoxyisoflavone (=compound 11) on the Running Performances on the Body Composition and on the Gastrocnemius-Plantaris-Soleus Muscle Group of Mice

20 male C57B1/6J mice were obtained from Jackson Laboratory (Bar Harbor, Me., USA) at the age of 5 weeks. All mice were administered a high-fat diet (Kliba#2154, Provimi Kliba AG, Kaiseraugst, Switzerland) for 8 weeks to induce obesity. Thereafter, the mice were randomly assigned into two groups:

| | |
|---|---|
| Group 1: | Control (high-fat diet) |
| Group 2: | 4′,7-Dimethoxyisoflavone (high-fat diet supplemented with |
| | 0.03% w/w 4′,7-Dimethoxyisoflavone (Alfa Aesar)) |

The duration of the supplementation was 9 weeks. During this period all mice received food and water ad libitum. At the end of the supplementation period, maximal running performance on a rodent treadmill (Technical & Scientific Equipment GmbH, Bad Homburg, Germany) was determined. Body composition was measured by quantitative magnetic resonance (Minispec MQ10, Bruker Optics GmbH, Faellanden, Switzerland) in conscious animals. At the end of the study, the animals were killed, blood was taken and the gastrocnemius-plantaris-soleus muscle group was excised and weighted.

Supplementation of mice with 4′,7-Dimethoxyisoflavone decreased body weight and the percentage of body fat mass while it increased the percentage of lean body mass compared to mice in the control group (Table 1). Furthermore, the weight of the gastrocnemius-plantaris-soleus muscle group relative to body weight was increased by 4′,7-Dimethoxyisoflavone consumption and maximal running distance increased by 6% (Table 1).

TABLE 1
Body weight, body fat mass, lean body mass, gastrocnemius-plantaris-
soleus weight and maximal running distance of control mice and
mice supplemented with 4′,7-Dimethoxyisoflavone.

| | | 4′,7-Dimethoxy- |
|---|---|---|
| | Control | isoflavone |
| Body weight (g) | 33.1 | 30.5 |
| Body fat mass (% of body weight) | 22.7 | 15.6 |
| Lean body mass (% of body weight) | 69.7 | 75.0 |
| Gastrocnemius-plantaris-soleus | 6.06 | 6.74 |
| weight (mg/g body weight) | | |
| Maximal running distance (m) | 2081 | 2201 |

Increased maximal running distance in a treadmill test demonstrates enhanced endurance is an indicator of improved oxidative capacity in skeletal muscle caused by the consumption of 4′,7-Dimethoxyisoflavone. Anatomically, this is reflected by an increased proportion of type I and type IIa muscle fibers. Furthermore, consumption of 4′,7-Dimethoxyisoflavone reduced body fat mass and increased lean body mass compared to control animals, thereby ameliorating the deleterious effects of the high-fat diet. This effect can be caused by increased fat oxidation in skeletal muscle due to a greater proportion of oxidative type I and type IIa muscle fibers.

Therefore, supplementation with 4′,7-Dimethoxyisoflavone enhances muscle function and endurance and improves the body shape as well as the muscle:fat ratio in mammals.

### Example 6

### Effect of Compounds 1-8 on Fat Metabolism including Fat Burning

Cell Culture

C2C12 subclone cells were obtained from Dr. Grimaldi, University of Nice, France. C2C12PPd cells were seeded in 24-well-plates with 0.1×106 to 0.15×106 and kept at 37° C. in maintenance medium (DMEM #41965 adjusted to 10% FBS, 2 mM L-Glutamine, 1 mM sodium pyruvate, 100 IU/ml penicillin and 100 μg/ml streptomycin). At 90-95% confluency, cells were washed with 1×PBS and changed to differentiation medium (DMEM #41965 adjusted to 3% FBS, 2 mM Glutamine and 1 mM sodium pyruvate). After 4 days of differentiation, cells were washed with 1×PBS and treated in treatment medium (DMEM #41965 adjusted to 2% BSA, 2 mM Glutamine and 100 IU/ml penicillin and 100 μg/ml streptomycin) with the test compounds. DMSO contents were adjusted to 0.5% final concentration.

After 24 h incubation with tested compounds, cells were washed with 1×PBS, harvested with 300 μl RLT buffer (Qiagen RNeasy Mini Kit #74106) in QIAshredder (Qiagen 79656) and stored at −20° C.

RNA Isolation and Analysis

Total RNA was extracted using the Qiagen RNeasy Mini Kit, including an in column DNase step (Qiagen #79254) according to the manufacture's protocol. RNA concentration was determined using RiboGreen® RNA quantification assay (Molecular Probes #R11490, Eugene, USA) according to the manufacturer's protocol. 200 ng of the total RNA were subject to first strand cDNA synthesis by reverse transcription using the Omniscript™ RT-kit (Qiagen #205113, Basel, CH) with 10 μM Random Primers (Promega #C1181) according to the manufacturer's instructions. The final volume was adjusted to 400 μl with DEPC-treated water (Ambion, Austin, Tex., USA) and stored at −20° C.

Gene Expression Analysis

Quantitative real-time TaqMan RT-PCR, based on the multiplex method, was used to quantify the expression levels of selected genes. In the quantitative TaqMan RT-PCR, 5 μl of the diluted cDNA was added to 20 μl of the reaction mixture, consisting of 12.5 μl TaqMan 2× Master Mix (PE biosystems, Rotkreuz, CH), 300 nM PCR primers (forward and reverse), and 100 nM TaqMan probe for the gene of interest. The reference gene used was 18S rRNA, with primers and probes at 50 nM and 100 nM, respectively. Probes for the gene of interest were labeled with FAM on the 5′ end and with Tamra on the 3′ end. The 18S rRNA probe was labeled with VIC on the 5′ end and with Tamra on the 3′ end. The oligonucleotide sequences for the primers and probes are shown in Table 2. Amplification was performed using an Abi-Prism 7700 Sequence Detector (PE Biosystems, Foster City, Calif., USA) in MicroAmp Optical 96-well reaction plates (PE Biosystems, Foster City, Calif., USA). The PCR amplification program consisted of 2 min at 50° C., 10 min at 95° C., and 40 cycles of 15 sec at 95° C. and 60 sec at 60° C. Threshold CT values were set at 0.05. The baseline start and stop values for the gene of interest were set at 3 and 15, respectively, and for the reference gene (18S rRNA) at 3 and 7, respectively. mRNA abundance was determined using the ΔCT method according to the manufacturer's protocol. Briefly, the ΔCT for the gene of interest was determined as the difference between the CT values for the reference gene and the gene of interest. Then, ΔΔCT was determined as the difference in dCT between the untreated control group and each of the treated groups. The fold induction for the gene of interest (i.e. the amount of mRNA for the gene of interest, normalized to an endogenous reference and relative to a calibrator) was determined as 2-ΔΔCT In graphs data is presented as a percentage.

Results

Genes in involved in fatty acid β-oxidation and lipid metabolism were checked using real-time RT-PCR. Fold of induction compared to DMSO control was shown in the following table 3.

TABLE 3

| Compound | mCD36 | mUCP2 | mCPT1b | mUCP3* | mACO1 | mLPL | mFABP3 |
|---|---|---|---|---|---|---|---|
| 1 | 2.93 | 2.46 | 1.33 | | 1.98 | 1.11 | 1.35 |
| 2 | 3.2 | 3.27 | 1.05 | | 1.81 | 1.91 | 1.63 |
| 3 | 10.16 | 4.25 | 3.64 | 4.73 | 1.54 | 3.5 | 4.76 |
| 4 | 15.56 | 7.55 | 5.08 | 5.95 | 1.74 | 3.31 | 6.29 |
| 5 | 1.91 | 2.33 | 4.19 | | 1.03 | 1.84 | 1.88 |
| 6 | 22.66 | 6.1 | 1.62 | 1.92 | 1.09 | 2.44 | 5.02 |
| 7 | 2.95 | 2.33 | 1.12 | | 1.03 | 1.06 | 2.16 |
| 8 | 9.49 | 4.68 | 1.29 | | 1.15 | 1.88 | 3.36 |

*only determined for the compounds with given value in the table.

Excess of fat in the peripheral tissue, such as adipose, skeletal muscle and liver result in insulin resistance, obesity, dyslipidemia and dysfunction of these organs. Skeletal muscle is one major site for glucose and lipid metabolism. Long term lipid accumulation in the muscle leads to not only dropped performance, but also chronic inflammation, which is associated with muscle protein degradation and aged related scarcopenia. Diseases associated with abnormal lipid metabolism, such as obesity and diabetes, result often in muscle wasting. Fatty acid, breakdown metabolites of triglyceride, is further oxidized in mitochondria, a process called TCA cycle, which generate ATP as energy source for the organism. Thus, increased fatty acid oxidation in the muscle would lead to increased lipid consumption, reduced fat storage, improved insulin sensitivity and improved muscle function.

A mouse skeletal muscle cell models was used to study effect of abovementioned natural compounds on expression of key regulators of lipid catabolism.

Lipoprotein lipase (LPL) hydrolyzes lipid on their transporters, lipoprotein, and free them for further catabolism. Compounds 3, 4 and 6 increased moderately expression of this enzyme. Furthermore fatty acid transporter and storage enzyme, such as CD36 and FABP3 (muscle specific form) were induced by most of compounds tested, with most remarkable effect seen with compounds 6, 4 and 3. The mitochondrial oxidation of long-chain fatty acids is initiated by the sequential action of carnitine palmitoyltransferase I (outer membrane and is detergent-labile) and carnitine palmitoyltransferase II (inner membrane). CPT I is the key enzyme in the carnitine-dependent transport across the mitochondrial inner membrane. Muscle specific form of this enzyme, CPT1b, was induced by compounds 4, 5 and 3 in the muscle cells. Acyl-CoA caboxylase (ACO1) is the first enzyme of the very long chain fatty acid beta-oxidation pathway, which catalyzes the desaturation of acyl-CoAs to 2-trans-enoyl-CoAs. It donates electrons directly to molecular oxygen, thereby producing hydrogen peroxide. Compounds 1, 4 and 2 increased expression of ACO1 by ˜2 fold, while other tested compound showed no effect. Last but not least, mitochondrial uncoupling proteins (UCP) are members of the larger family of mitochondrial anion carrier proteins and play important role in thermogenesis. UCPs facilitate the transfer of anions from the inner to the outer mitochondrial membrane and the return transfer of protons from the outer to the inner mitochondrial membrane. skeletal muscle has highest expression level of UCPs. We observed positive effect on UCP2 from all tested compounds and increased UCP3 expression from compounds 4, 6 and 3.

In summary, compounds 1-8 showed selective activation of a panel of genes involved in fatty acid oxidation and mitochondrial uncoupling in the muscle cells, indicating their function in modulating lipid metabolism and muscle function.

TABLE 2

| | | | | |
|---|---|---|---|---|
| Gene | Genbank No. | Primer forward | Primer reverse | Probe |

| | | | | |
|---|---|---|---|---|
| m ACC2 | BC022940 | CGACCTGCACGACACCC | TGCGGGCAGTCTTCCATT | CCACATGCTGGAGAAAGGAATCATTTCT |
| | | | | GA |
| m ACO1 | AF006688 | TGGCCAAGGCGACCTG | AAGCCTTCAGCCCAGCTGT | TGAGCTGCCTGAGCTTCATGCCC |
| m CD36 | AK052825 | AGACCTTACATTGTACCTATACTGT | TTGTGTTTTGAACATTTCTGCTTTT | AAATGAGACTGGGACCATTGGTGATGAA |
| | | GGC | | |
| m CPT1b | AF017174 | CCAATCATCTGGGTGCTGG | TAAGAGACCCCGTAGCCATCAT | TGGCTTTGGTCCCGTGGCG |
| m FABP3 | U02883 | ACCGCCCGCTCCTCT | TGAGGCAGCATGGTGCTG | CCAACTGGCCACCCCTCAGC |
| m LPL | BC003305 | GTGGCCGAGAGCGAGAAC | AAGAAGGAGTAGGTTTTATTTGTGG | TTCCCTTCACCCTGCCCGAGG |
| | | | AA | |
| m PPARd | BC070398 | GCGGCCATCATTCTGTGTG | CTGGATGGCTTCTACCTGGG | AGACCGGCCAGGCCTCATGAATG |
| m UCP2 | AK035298 | AGGCCTCAGCCTGAGACCT | GCAAGACGAGACAGAGGAACTC | AAAGCAGCCTCCAGAACTCCGGC |
| m UCP3 | AB010742 | GTCTCACCTGTTTACTGACAACTTC | CACCACTGTGGCACAGAA | TCACTTTGTCTCTGCCTTTGGAGCTGG |

1. Use of a compound of the general formula Ig, 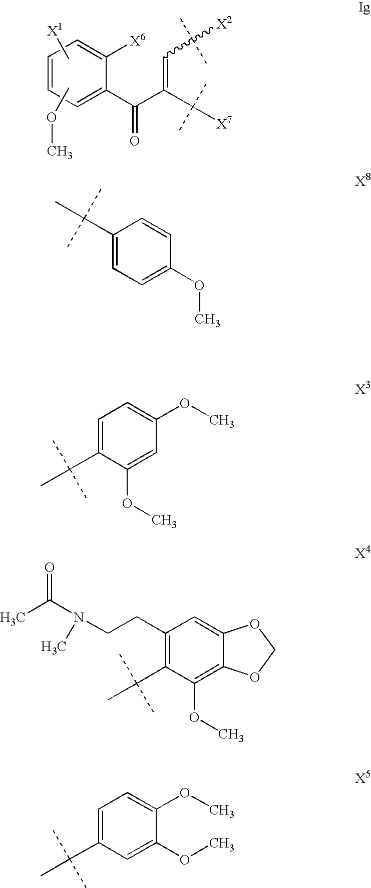
with X¹ being H or CH₃, and
X² being X³, X⁴ or X⁵; or X² and X⁶ together forming an oxygen bond; and
X⁷ being H or X⁸,
with the proviso that X⁶═X¹ and X⁷═H if X²═X³, X⁴ or X⁵; and
with the further proviso that X¹═H and X⁷═X⁸ if X² and X⁶ together form an oxygen bond,
for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

2. The use according to claim 1, characterized in that the compound of the general formula Ig is selected from the group consisting of compounds 3, 4, 6 and 11.

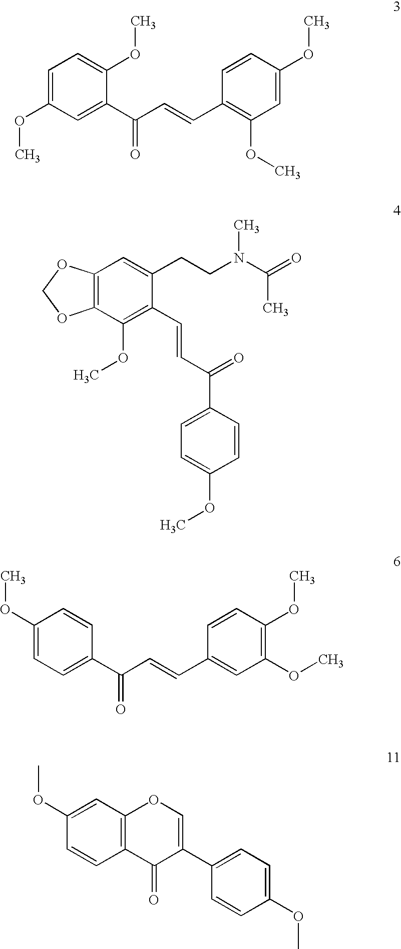

3. The use according to claim 1, characterized in that the compound of the general formula Ig is compound 11.

4. A composition, preferably a dietary, body care or pharmaceutical composition, containing at least one compound of the general formula Ig according to.

5. The composition according to claim 4, wherein the composition is a dietary composition in form of food such as dairy products (e.g. yoghurts), in form of fortified food such as cereal bars and bakery items such as cakes and cookies, in form of dietary supplements such as tablets, pills, granules, dragées, capsules, and effervescent formulations, in form of non-alcoholic drinks such as soft drinks, sport drinks, fruit juices, lemonades, teas and milk based drinks, in form of liquid food such as soups and dairy products (muesli drinks).

6. Use of the composition according to claim 4 for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

7. A method for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans, said method comprising administering an effective dose of a compound of the general formula Ig as defined in to mammals including humans which are in need thereof.

8. The method according to claim 7, wherein the mammal is a human, a pet animal or a farm animal.

9. Use of a compound of the general formula I or Ie, 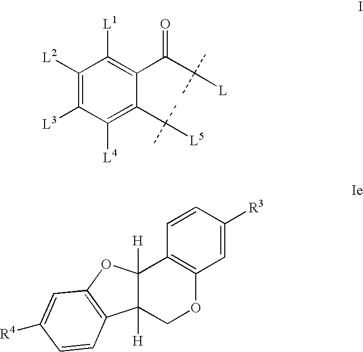
wherein L is either A or B when L⁵ is hydrogen or R⁵, or 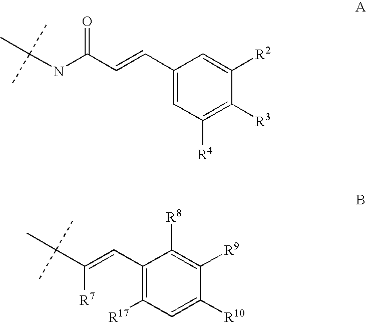
wherein L and L⁵ form the residue C or D, 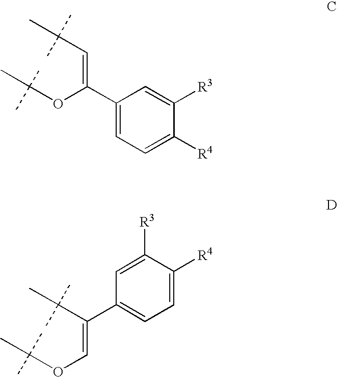
and wherein
L¹ is H, OH or R²; L² is H; L³ is H, OH or R⁶; L⁴ is H;
preferably L¹ is OH and L², L³ and L⁴ is H, when L is A;
preferably L¹, L² and L⁴ are H, L³ is R⁶ and L⁵ is R⁵ when L is B;
preferably L², L⁴ is H, L¹ is R² and L³ is R⁶ when L and L⁵ form together the residue C;
preferably L², L⁴ is H, L¹ is R² and L³ is R⁶ when L and L⁵ form together the residue D;
R² is H, OH or C₁₋₆-alkyloxy; R³, R⁴ and R⁶ are independently from each other OH or C₁₋₆-alkyloxy;
R⁵ is H or C₁₋₆-alkyloxy; R⁷ is H; or R⁵ and R⁷ are together —O—; R⁸, R¹⁰ is C₁₋₆-alkyloxy;
R⁹ is H, C₁₋₆-alkyloxy or cinnamyloxy; or R⁹ and R¹⁰ form together a group O—(CH₂)ₓ—O with x=1, 2, 3; R¹⁷ is (N—C₁l₆-acyloxy, N—C₁₋₆-alkyloxy)-y-amino-C_{y}-alkyl with y=1-6, for treating muscular disorders including muscle wasting and associated disorders such as sarcopenia, cachexia, muscular damage, muscular dystrophies and muscular fatigue, for improving muscle function and endurance, for the treatment of disorders connected to impaired lipid metabolism, impaired glucose metabolism, impaired insulin action and for the treatment of obesity, overweight conditions, eating disorders such as bulimia and anorexia nervosa, for bodyshaping, for improving the muscle:fat ratio, for the treatment of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, dermatitis, and allergy, for accelerating skin wound healing, for treating respiratory disorders such as asthma, chronic obstructive pulmonary disease (COPD), allergic diseases, and/or for treating bone disorders such as osteoporosis and osteopenia.

10. The use according to claim 9, characterized in that the compound of the general formula I is a compound of the formula Ia, Ib, Ic or Id, 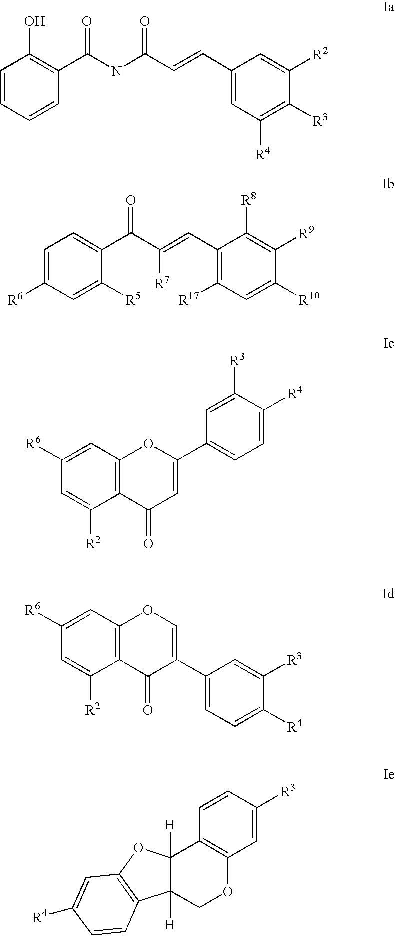
wherein
R² is H, OH or C₁₋₆-alkyloxy; R³, R⁴ and R⁶ are independently from each other OH or C₁₋₆-alkyloxy;
R⁵ is H or C₁₋₆-alkyloxy; R⁷ is H; or R⁵ and R⁷ are together —O—; R⁸ and R¹⁰ are independently from each other C₁₋₆-alkyloxy;
R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6.

11. The use according to claim 9, wherein the substituents of the compounds are as follows: R² is H, OH or methoxy; and/or R³, R⁴ and R⁶ are independently from each other OH or methoxy; and/or R⁵ is H or methoxy; or R⁷ is H; or R⁵ and R⁷ are together —O—; R⁸ and/or R¹⁰ are methoxy; or R⁹ is H, methoxy or cinnamyloxy; or R⁹ and R¹⁰ form together a group O—(CH₂)ₓ—O with x=1, 2; and/or R¹⁷ is N-acyl,N-methyl)-2-aminoethyl.

12. The use according to claim 9, wherein the compound of the general formula I is selected from the group consisting of the compounds 1-12, wherein compound 1 is the compound of the general formula Ia, wherein R1=R3=R4=OH and R2=H; compound 2 is the compound of the general formula Ia, wherein R1=OH, R2=R3=R4=methoxy; compound 3 is the compound of the general formula Ib, wherein R5=R6=R8=R10=methoxy and R7=R9=R17=H; compound 4 is the compound of the general formula Ib, wherein R5=R7=H, R6=R8=methoxy, R9 and R10 form together —O—CH₂—O— and R17=N-acyl,N-methyl)-2-aminoethyl; compound 5 is the compound of the general formula Ib, wherein R5=R7=R8=R10=R17=H, R6=methoxy and R9=cinnamyloxy; compound 6 is the compound of the general formula Ib, wherein R5=R7=R8=R17=H, R6=R9=R10=methoxy; compound 7 is the compound of the general formula Ib, wherein R8=R9=R17=H, R6=OH, R5 and R7 are together O, R10=methoxy; compound 8 is the compound of the general formula Ib, wherein R5=R7=R9=R17=H, R6=R8=R10=methoxy; compound 9 is the compound of the general formula Ic, wherein R3=R4=OH, and R2=R6=H; compound 10 is the compound of the general formula Ic, wherein R2=R3=R6=OH, and R4=methoxy; compound 11 is the compound of the general formula Id, wherein R2=R3=H and R4=R6=methoxy; and compound 12 is the compound of the general formula Ie, wherein R3=R4=methoxy and the two hydrogens are cis to each other.

13. A composition, preferably a dietary, body care or pharmaceutical composition, containing at least one compound of the general formula I or Ie, 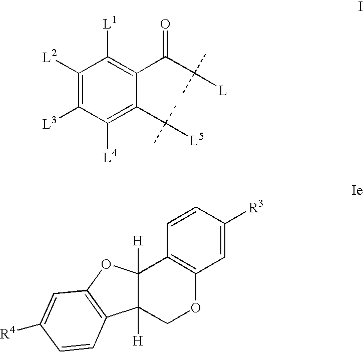
wherein L is either A or B when L⁵ is hydrogen or R⁵, or 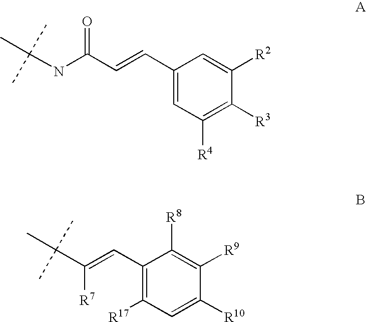
wherein L and L⁵ form the residue C or D, 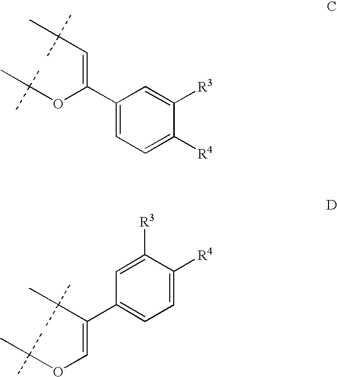
and wherein
L¹ is H, OH or R²; L² is H; L³ is H, OH or R⁶; L⁴ is H;
preferably L¹ is OH and L², L³ and L⁴ is H, when L is A;
preferably L¹, L² and L⁴ are H, L³ is R⁶ and L⁵ is R⁵ when L is B;
preferably L², L⁴ is H, L¹ is R² and L³ is R⁶ when L and L⁵ form together the residue C;
preferably L², L⁴ is H, L¹ is R² and L³ is R⁶ when L and L⁵ form together the residue D;
R² is H, OH or C₁₋₆-alkyloxy; R³, R⁴ and R⁶ are independently from each other OH or C₁₋₆-alkyloxy;
R⁵ is H or C₁₋₆-alkyloxy; R⁷ is H; or R⁵ and R⁷ are together —O—; R⁸, R¹⁰ is C₁₋₆-alkyloxy;
R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6.

14. The composition according to claim 13, characterized in that the compound of the general formula I is a compound of the formula Ia, Ib, Ic or Id, 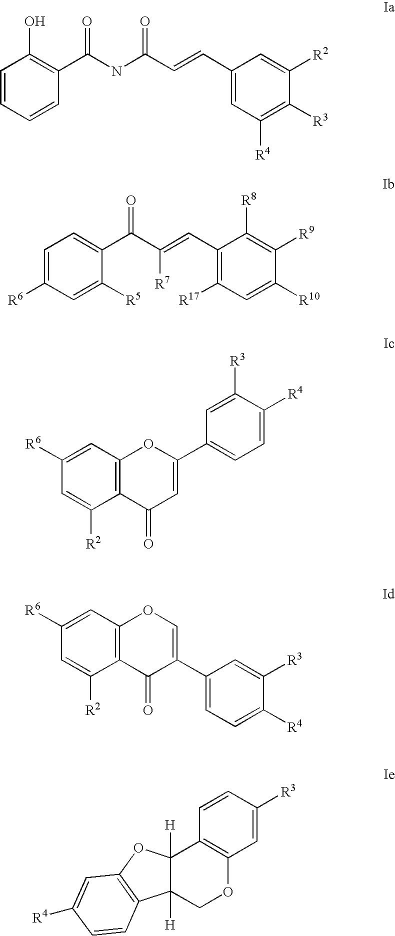
wherein
R² is H, OH or C₁₋₆-alkyloxy; R³, R⁴ and R⁶ are independently from each other OH or C₁₋₆-alkyloxy;
R⁵ is H or C₁₋₆-alkyloxy; R⁷ is H; or R⁵ and R⁷ are together —O—; R⁸ and R¹⁰ are independently from each other C₁₋₆-alkyloxy;
R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6.

15. The composition according to claim 13, preferably the dietary, body care or pharmaceutical composition, wherein R² is H, OH or methoxy; and/or R³, R⁴ and R⁶ are independently from each other OH or methoxy; and/or R⁵ is H or methoxy; or R⁷ is H; or R⁵ and R⁷ are together —O—; R⁸ and/or R¹⁰ are methoxy; or R⁹ is H, methoxy or cinnamyloxy; or R⁹ and R¹⁰ form together a group O—(CH₂)ₓ—O with x=1, 2; and/or R¹⁷ is N-acyl,N-methyl)-2-aminoethyl.

16. The composition according to claim 13, preferably the dietary, body care or pharmaceutical composition, characterized in that the compound of the general formula I is selected from the group consisting of the compound of the general formula Ia, wherein R1=R3=R4=OH and R2=H (=compound 1); the compound of the general formula Ia, wherein R1=OH, R2=R3=R4=methoxy (=compound 2); the compound of the general formula Ib, wherein R5=R6=R8=R10=methoxy and R7=R9=R17=H (=compound 3); the compound of the general formula Ib, wherein R5=R7=H, R6=R8=methoxy, R9 and R10 form together —O—CH₂—O— and R17=N-acyl,N-methyl)-2-aminoethyl (=compound 4); the compound of the general formula Ib, wherein R5=R7=R8=R10=R17=H, R6=methoxy and R9=cinnamyloxy (=compound 5); the compound of the general formula Ib, wherein R5=R7=R8=R17=H, R6=R9=R10=methoxy (=compound 6); the compound of the general formula Ib, wherein R8=R9=R17=H, R6=OH, R5 and R7 are together O, R10=methoxy (=compound 7); the compound of the general formula Ib, wherein R5=R7=R9=R17=H, R6=R8=R10=methoxy (=compound 8); the compound of the general formula Ic, wherein R3=R4=OH, and R2=R6=H (=compound 9); the compound of the general formula Ic, wherein R2=R3=R6=OH, and R4=methoxy (=compound 10); the compound of the general formula Id, wherein R2=R3=H and R4=R6=methoxy (=compound 11); and the compound of the general formula Ie, wherein R3=R4=methoxy and the two hydrogens are cis to each other (=compound 12).

17. The composition according to being a dietary composition in form of food such as dairy products (e.g. yoghurts), in form of fortified food such as cereal bars and bakery items such as cakes and cookies, in form of dietary supplements such as tablets, pills, granules, dragées, capsules, and effervescent formulations, in form of non-alcoholic drinks such as soft drinks, sport drinks, fruit juices, lemonades, teas and milk based drinks, in form of liquid food such as soups and dairy products (muesli drinks).

18. Use of the dietary or pharmaceutical composition according to for treating muscular disorders including muscle wasting and associated disorders such as sarcopenia, cachexia, muscular damage, muscular dystrophies and muscular fatigue, for improving muscle function and endurance, for the treatment of disorders connected to impaired lipid metabolism, impaired glucose metabolism, impaired insulin action and for the treatment of obesity, overweight conditions, eating disorders such as bulimia and anorexia nervosa, for bodyshaping, for improving the muscle:fat ratio, for the treatment of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, dermatitis, and allergy, for accelerating skin wound healing, for treating respiratory disorders such as asthma, chronic obstructive pulmonary disease (COPD), allergic diseases, and/or for treating bone disorders such as osteoporosis and osteopenia.

19. Compound of the general formula I or Ie as defined in claim 9 for use as medicament.

20. The compound for use as medicament according to claim 19, characterized in that the compound of the general formula I is selected from the group consisting of the compound of the general formula Ia, wherein R1=R3=R4=OH and R2=H (=compound 1); the compound of the general formula Ia, wherein R1=OH, R2=R3=R4=methoxy (=compound 2); the compound of the general formula Ib, wherein R5=R6=R8=R10=methoxy and R7=R9=R17=H (=compound 3); the compound of the general formula Ib, wherein R5=R7=H, R6=R8=methoxy, R9 and R10 form together —O—CH₂—O— and R17=N-acyl,N-methyl)-2-aminoethyl (=compound 4); the compound of the general formula Ib, wherein R5=R7=R8=R10=R17=H, R6=methoxy and R9=cinnamyloxy (=compound 5); the compound of the general formula Ib, wherein R5=R7=R8=R17=H, R6=R9=R10=methoxy (=compound 6); the compound of the general formula Ib, wherein R8=R9=R17=H, R6=OH, R5 and R7 are together O, R10=methoxy (=compound 7); the compound of the general formula Ib, wherein R5=R7=R9=R17=H, R6=R8=R10=methoxy (=compound 8); the compound of the general formula Ic, wherein R3=R4=OH, and R2=R6=H (=compound 9); the compound of the general formula Ic, wherein R2=R3=R6=OH, and R4=methoxy (=compound 10); the compound of the general formula Id, wherein R2=R3=H and R4=R6=methoxy (=compound 11); and the compound of the general formula Ie, wherein R3=R4=methoxy and the two hydrogens are cis to each other (=compound 12).

21. The compound for use as medicament according to claim 19, wherein the medicament is used for the treatment of muscular disorders including muscle wasting and associated disorders such as sarcopenia, cachexia, muscular damage, muscular dystrophies and muscular fatigue, for improving muscle function and endurance, for the treatment of disorders connected to impaired lipid metabolism, impaired glucose metabolism, impaired insulin action and for the treatment of obesity, overweight conditions, eating disorders such as bulimia and anorexia nervosa, for bodyshaping, for improving the muscle:fat ratio, for the treatment of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, dermatitis, and allergy, for accelerating skin wound healing, for treating respiratory disorders such as asthma, chronic obstructive pulmonary disease (COPD), allergic diseases, and/or for treating bone disorders such as osteoporosis and osteopenia.

22. Use of a compound of the general formula I or Ie as defined in claim 9, preferably of a compound as defined in, for the manufacture of a composition for the treatment of muscular disorders including muscle wasting and associated disorders such as sarcopenia, cachexia, muscular damage, muscular dystrophies and muscular fatigue, for improving muscle function and endurance, for the treatment of disorders connected to impaired lipid metabolism, impaired glucose metabolism, impaired insulin action and for the treatment of obesity, overweight conditions, eating disorders such as bulimia and anorexia nervosa, for bodyshaping, for improving the muscle:fat ratio, for the treatment of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, dermatitis, and allergy, for accelerating skin wound healing, for treating respiratory disorders such as asthma, chronic obstructive pulmonary disease (COPD), allergic diseases, and/or for treating bone disorders such as osteoporosis and osteopenia.

23. The compound according to claim 19, wherein the medicament and the composition, respectively, is used for enhancing physical performance, enhancing endurance capacity, increasing muscle mass, maintaining muscle performance and/or muscle strength and/or muscle mass and/or muscle function, improving bone health and function, preventing muscle loss, enhance muscle recovery, reducing muscle fatigue, increasing fat oxidation, improving energy balance, improving blood lipid profile, decreasing bad cholesterol (LDL) and increasing good cholesterol (HDL), protecting against obesity, promoting fat oxidation, maintaining healthy glucose and cholesterol levels, preventing cardiovascular diseases, improving insulin sensitivity, maintaining endothelial function, and/or for improving joint function, reducing arthrthitic pain and swelling, improving skin wound healing, and/or for improving inflammatory skin conditions.

24. A method for the treatment and/or prevention of muscular disorders including muscle wasting and associated disorders such as sarcopenia, cachexia, muscular damage, muscular dystrophies and muscular fatigue, for the improvement of muscle function and endurance, for the treatment of disorders connected to impaired lipid metabolism, impaired glucose metabolism, impaired insulin action and for the treatment of obesity, overweight conditions, eating disorders such as bulimia and anorexia nervosa, for bodyshaping, for improving the muscle:fat ratio, for the treatment of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, dermatitis, and allergy, and for accelerating skin wound healing, respiratory disorders such as asthma, chronic obstructive pulmonary disease (COPD), allergic diseases, and/or bone disorders such as osteoporosis and osteopenia, in mammals including humans, said method comprising administering an effective dose of a compound of the general formula I or Ie as defined in claim 9, preferably of a compound as defined in, to mammals including humans which are in need thereof.

25. The method according to claim 24, wherein the mammal is a human, a pet animal or a farm animal.

26. A compound of the general formula I or Ie, 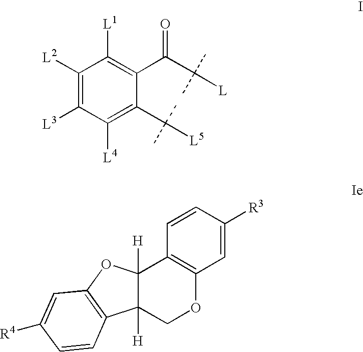
wherein L is either A or B when L⁵ is hydrogen or R⁵, or 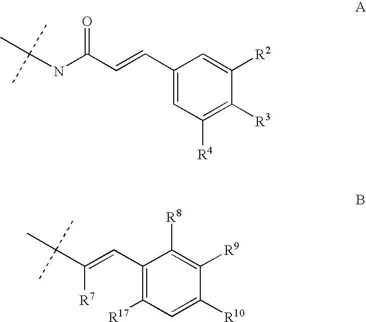
wherein L and L⁵ form the residue C or D, 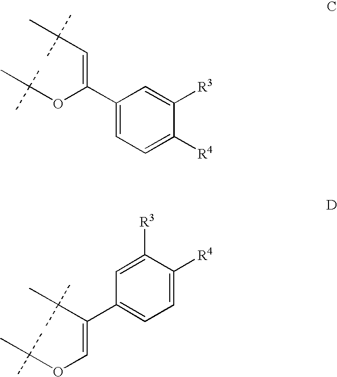
and wherein
L¹ is H, OH or R²; L² is H; L³ is H, OH or R⁶; L⁴ is H;
preferably L¹ is OH and L², L³ and L⁴ is H, when L is A;
preferably L¹, L² and L⁴ are H, L³ is R⁶ and L is R⁵ when L is B;
preferably L², L⁴ is H, L¹ is R² and L³ is R⁶ when L and L⁵ form together the residue C;
preferably L², L⁴ is H, L¹ is R² and L³ is R⁶ when L and L⁵ form together the residue D;
R² is H, OH or C₁₋₆-alkyloxy; R³, R⁴ and R⁶ are independently from each other OH or C₁₋₆-alkyloxy;
R⁵ is H or C₁₋₆-alkyloxy; R⁷ is H; or R⁵ and R⁷ are together —O—; R⁸, R¹⁰ is C₁₋₆-alkyloxy;
R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6, for treating muscular disorders including muscle wasting and associated disorders such as sarcopenia, cachexia, muscular damage, muscular dystrophies and muscular fatigue, for improving muscle function and endurance, for the treatment of disorders connected to impaired lipid metabolism, impaired glucose metabolism, impaired insulin action and for the treatment of obesity, overweight conditions, eating disorders such as bulimia and anorexia nervosa, for bodyshaping, for improving the muscle:fat ratio, for the treatment of cardiovascular diseases, atherosclerosis, vascular diseases, heart failure, inflammatory disorders such as arthritis, rheumatoid arthritis, osteoarthritis, gout, gastrointestinal disorders such as inflammatory bowel syndrome, Crohn's disease, gastritis, irritable bowel syndrome and ulcerative colitis, skin related conditions such as psoriasis, eczema, burns, dermatitis, and allergy, for accelerating skin wound healing, for treating respiratory disorders such as asthma, chronic obstructive pulmonary disease (COPD), allergic diseases, and/or for treating bone disorders such as osteoporosis and osteopenia.

27. The compound according to claim 26, characterized in that the compound of the general formula I is a compound of the formula Ia, Ib, Ic or Id, 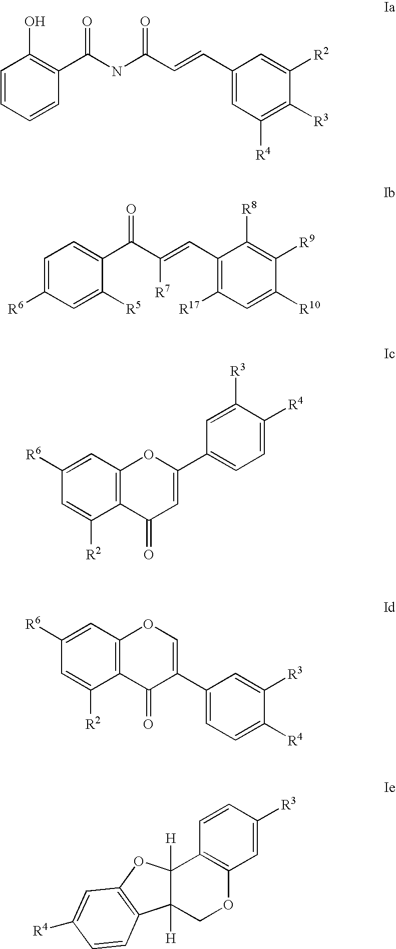
wherein
R² is H, OH or C₁₋₆-alkyloxy; R³, R⁴ and R⁶ are independently from each other OH or C₁₋₆-alkyloxy;
R⁵ is H or C₁₋₆-alkyloxy; R⁷ is H; or R⁵ and R⁷ are together —O—; R⁸ and R¹⁰ are independently from each other C₁₋₆-alkyloxy;
R⁹ is H, C₁₋₆-alkyloxy or cinnamyloxy; or R⁹ and R¹⁰ form together a group O—(CH₂)ₓ—O with x=1, 2, 3; R¹⁷ is (N—C₁₋₆-acyloxy, N—C₁₋₆-alkyloxy)-y-amino-C_{y}-alkyl with y=1-6.

28. The compound according to claim 26, wherein the substituents of the compounds are as follows: R² is H, OH or methoxy; and/or R³, R⁴ and R⁶ are independently from each other OH or methoxy; and/or R⁵ is H or methoxy; or R⁷ is H; or R⁵ and R⁷ are together —O—; R⁸ and/or R¹⁰ are methoxy; or R⁹ is H, methoxy or cinnamyloxy; or R⁹ and R¹⁰ form together a group O—(CH₂)ₓ—O with x=1, 2; and/or R¹⁷ is N-acyl,N-methyl)-2-aminoethyl.

29. The compound according to claim 26, wherein the compound of the general formula I is selected from the group consisting of the compounds 1-12, wherein compound 1 is the compound of the general formula Ia, wherein R1=R3=R4=OH and R2=H; compound 2 is the compound of the general formula Ia, wherein R1=OH, R2=R3=R4=methoxy; compound 3 is the compound of the general formula Ib, wherein R5=R6=R8=R10=methoxy and R7=R9=R17=H; compound 4 is the compound of the general formula Ib, wherein R5=R7=H, R6=R8=methoxy, R9 and R10 form together —O—CH₂—O— and R17=N-acyl,N-methyl)-2-aminoethyl; compound 5 is the compound of the general formula Ib, wherein R5=R7=R8=R10=R17=H, R6=methoxy and R9=cinnamyloxy; compound 6 is the compound of the general formula Ib, wherein R5=R7=R8=R17=H, R6=R9=R10=methoxy; compound 7 is the compound of the general formula Ib, wherein R8=R9=R17=H, R6=OH, R5 and R7 are together O, R10=methoxy; compound 8 is the compound of the general formula Ib, wherein R5=R7=R9=R17=H, R6=R8=R10=methoxy; compound 9 is the compound of the general formula Ic, wherein R3=R4=OH, and R2=R6=H; compound 10 is the compound of the general formula Ic, wherein R2=R3=R6=OH, and R4=methoxy; compound 11 is the compound of the general formula Id, wherein R2=R3=H and R4=R6=methoxy; and compound 12 is the compound of the general formula Ie, wherein R3=R4=methoxy and the two hydrogens are cis to each other.

30. Use of 4′,7-dimethoxyisoflavone for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

31. 4′,7-dimethoxyisoflavone for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

32. Use of 4′,7-dimethoxyisoflavone for the manufacture of a composition for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans.

33. A method for treating muscular disorders including muscle wasting and associated disorders such as sarcopenia, cachexia, muscular damage, muscular dystrophies and muscular fatigue, for improving muscle function and endurance, for enhancing physical performance, for enhancing endurance capacity, for increasing muscle mass, for preventing muscle loss, for enhancing muscle recovery, for reducing muscle fatigue, for improving energy balance, for the maintenance of muscle performance and/or muscle strength and/or muscle mass and/or muscle function, and/or for improving the body shape and/or for improving the muscle:fat ratio in mammals including humans, said method comprising administering an effective dose of 4′,7-dimethoxyisoflavone to mammals including humans which are in need thereof.
